(19) United States
(12) Patent Application Publication
(10) Pub. No.: US 2010/0267753 A1
(43) Pub. Date: Oct. 21, 2010

(54) METHODS OF TREATING DISORDERS ASSOCIATED WITH PROTEIN KINASE CK2 ACTIVITY

(76) Inventors: Mustapha HADDACH, San Diego, CA (US); Sean O'Brien, Carlsbad, CA (US); Kenna Anderes, San Diego, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/762,038

(22) Filed: Apr. 16, 2010

Related U.S. Application Data
(60) Provisional application No. 61/170,468, filed on Apr. 17, 2009.
(60) Provisional application No. 61/240,165, filed on Sep. 4, 2009.
(60) Provisional application No. 61/242,227, filed on Sep. 14, 2009.
(60) Provisional application No. 61/297,669, filed on Jan. 22, 2010.

(51) Int. Cl. A61K 31/4375; A61K 31/519; A61P 31/16; A61P 25/00; A61P 29/00; A61P 7/00; A61P 21/00; A61P 33/02; A61P 35/02; A61P 31/12; A61P 25/28; A61P 25/16; A61P 31/18; A61P 31/22

(52) U.S. Cl. 514/268; 514/293

(57) ABSTRACT

The invention provides methods for the treatment or amelioration of disorders associated with undesired activity of protein kinase CK2, using compounds of Formula (I)

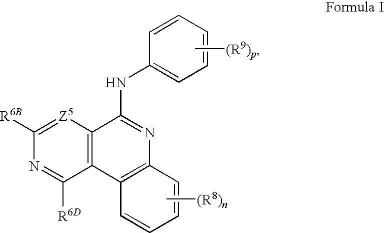

that are potent, selective inhibitors of CK2, and pharmaceutical compositions of such compounds, wherein Z⁵, R^{6B}, R^{6D}, R⁸, n, R⁹ and p are defined as further described herein,

### CROSS-REFERENCE TO RELATED APPLICATIONS

This application claims priority from U.S. Provisional Application Ser. No. 61/170,468, filed Apr. 17, 2009; U.S. Provisional Application Ser. No. 61/240,165, filed Sep. 4, 2009; U.S. Provisional Application Ser. No. 61/242,227, filed Sep. 14, 2009; and U.S. Provisional Application Ser. No. 61/297,669, filed Jan. 22, 2010. The contents of these applications are incorporated herein by reference in their entirety.

This application is related to PCT/US2007/077464, PCT/US2008/074820, PCT/US2009/035609, and U.S. Provisional Patent Application Ser. No. 61/143,282. The contents of these applications are incorporated herein by reference.

### TECHNICAL FIELD

The invention relates in part to molecules having certain biological activities that include, but are not limited to, inhibiting cell proliferation, and modulating serine-threonine protein kinase activity. Molecules of the invention modulate protein kinase CK2 (casein kinase II, or CK2) activity in particular, and are thus useful for treatment of disorders characterized by excessive, undesired or aberrant CK2 activity. The invention also relates in part to methods to use such molecules for the treatment or amelioration of disorders associated with undesired activity of CK2, and conditions where inhibition of CK2 is therapeutically useful such as in the treatment of pain, cancers and inflammatory conditions, as well as certain infectious disorders.

### BACKGROUND

Protein kinase CK2 (formerly called Casein kinase II, referred to herein as “CK2”) is a ubiquitous and highly conserved protein serine/threonine kinase. The holoenzyme is typically found in tetrameric complexes consisting of two catalytic (alpha and/or alpha') subunits and two regulatory (beta) subunits. CK2 has a number of physiological targets and participates in a complex series of cellular functions including the maintenance of cell viability. The level of CK2 in normal cells is tightly regulated, and it has long been considered to play a role in cell growth and proliferation Inhibitors of CK2 that are useful for treating certain types of cancers are described in PCT/US2007/077464, PCT/US2008/074820, and PCT/US2009/035609.

Both the prevalence and the importance of CK2 suggest it is an ancient enzyme on the evolutionary scale, as does an evolutionary analysis of its sequence; its longevity may explain why it has become important in so many biochemical processes, and why CK2 from hosts have even been co-opted by infectious pathogens (e.g., viruses, protozoa) as an integral part of their survival and life cycle biochemical systems. These same characteristics explain why inhibitors of CK2 are believed to be useful in a variety of medical treatments as discussed herein. Because it is central to many biological processes, as summarized by Guerra & Issinger, Curr. Med. Chem., 2008, 15:1870-1886, inhibitors of CK2, including the compounds described herein, should be useful in the treatment of a variety of diseases and disorders.

Cancerous cells show an elevation of CK2, and recent evidence suggests that CK2 exerts potent suppression of apoptosis in cells by protecting regulatory proteins from caspase-mediated degradation. The anti-apoptotic function of CK2 may contribute to its ability to participate in transformation and tumorigenesis. In particular, CK2 has been shown to be associated with acute and chronic myelogenous leukemia, lymphoma and multiple myeloma. In addition, enhanced CK2 activity has been observed in solid tumors of the colon, rectum and breast, squamous cell carcinomas of the lung and of the head and neck (SCCHN), adenocarcinomas of the lung, colon, rectum, kidney, breast, and prostate Inhibition of CK2 by a small molecule is reported to induce apoptosis of pancreatic cancer cells, and hepatocellular carcinoma cells (HegG2, Hep3, HeLa cancer cell lines); and CK2 inhibitors dramatically sensitized RMS (Rhabdomyosarcoma) tumors toward apoptosis induced by TRAIL. Thus an inhibitor of CK2 alone, or in combination with TRAIL or a ligand for the TRAIL receptor, would be useful to treat RMS, the most common soft-tissue sarcoma in children. In addition, elevated CK2 has been found to be highly correlated with aggressiveness of neoplasias, and treatment with a CK2 inhibitor of the invention should thus reduce tendency of benign lesions to advance into malignant ones, or for malignant ones to metastasize.

Unlike other kinases and signaling pathways, where mutations are often associated with structural changes that cause loss of regulatory control, increased CK2 activity level appears to be generally caused by upregulation or overexpression of the active protein rather than by changes that affect activation levels. Guerra and Issinger postulate this may be due to regulation by aggregation, since activity levels do not correlate well with mRNA levels. Excessive activity of CK2 has been shown in many cancers, including SCCHN tumors, lung tumors, breast tumors, and others. Id.

Elevated CK2 activity in colorectal carcinomas was shown to correlate with increased malignancy. Aberrant expression and activity of CK2 have been reported to promote increase nuclear levels of NF-kappaB in breast cancer cells. CK2 activity is markedly increased in patients with AML and CML during blast crisis, indicating that an inhibitor of CK2 should be particularly effective in these conditions. Multiple myeloma cell survival has been shown to rely on high activity of CK2, and inhibitors of CK2 were cytotoxic to MM cells. Similarly, a CK2 inhibitor inhibited growth of murine p190 lymphoma cells. Its interaction with Bcr/Abl has been reported to play an important role in proliferation of Bcr/Abl expressing cells, indicating inhibitors of CK2 may be useful in treatment of Bcr/Abl-positive leukemias Inhibitors of CK2 have been shown to inhibit progression of skin papillomas, prostate and breast cancer xenografts in mice, and to prolong survival of transgenic mice that express prostate-promoters. Id.

The role of CK2 in various non-cancer disease processes has been recently reviewed. See Guerra & Issinger, Curr. Med. Chem., 2008, 15:1870-1886. Increasing evidence indicates that CK2 is involved in critical diseases of the central nervous system, including, for example, Alzheimer's disease, Parkinson's disease, and rare neurodegenerative disorders such as Guam-Parkinson dementia, chromosome 18 deletion syndrome, progressive supranuclear palsy, Kuf's disease, or Pick's disease. It is suggested that selective CK2-mediated phosphorylation of tau proteins may be involved in progressive neurodegeneration of Alzheimer's. In addition, recent studies suggest that CK2 plays a role in memory impairment and brain ischemia, the latter effect apparently being mediated by CK2's regulatory effect on the PI3K survival pathways.

CK2 has also been shown to be involved in the modulation of inflammatory disorders, for example, acute or chronic inflammatory pain, glomerulonephritis, and autoimmune diseases, including, e.g., multiple sclerosis (MS), systemic lupus erythematosus, rheumatoid arthritis, and juvenile arthritis. It positively regulates the function of the serotonin 5-HT3 receptor channel, activates heme oxygenase type 2, and enhances the activity of neuronal nitric oxide synthase. A selective CK2 inhibitor was reported to strongly reduce pain response of mice when administered to spinal cord tissue prior to pain testing. It phosphorylates secretory type IIA phospholipase A2 from synovial fluid of RA patients, and modulates secretion of DEK (a nuclear DNA-binding protein), which is a proinflammatory molecule found in synovial fluid of patients with juvenile arthritis. Thus inhibition of CK2 is expected to control progression of inflammatory pathologies such as those described here, and the inhibitors disclosed herein have been shown to effectively treat pain in animal models.

Protein kinase CK2 has also been shown to play a role in disorders of the vascular system, such as, e.g., atherosclerosis, laminar shear stress, and hypoxia. CK2 has also been shown to play a role in disorders of skeletal muscle and bone tissue, such as cardiomyocyte hypertrophy, impaired insulin signaling and bone tissue mineralization. In one study, inhibitors of CK2 were effective at slowing angiogenesis induced by growth factor in cultured cells. Moreover, in a retinopathy model, a CK2 inhibitor combined with octreotide (a somatostatin analog) reduced neovascular tufts; thus the CK2 inhibitors described herein would be effective in combination with a somatostatin analog to treat retinopathy.

CK2 has also been shown to phosphorylate GSK, troponin and myosin light chain; thus it is important in skeletal muscle and bone tissue physiology, and is linked to diseases affecting muscle tissue.

Evidence suggests that CK2 is also involved in the development and life cycle regulation of protozoal parasites, such as, for example, Theileria parva, Trypanosoma cruzi, Leishmania donovani, Herpetomonas muscarum muscarum, Plasmodium falciparum, Trypanosoma brucei, Toxoplasma gondii and Schistosoma mansoni. Numerous studies have confirmed the role of CK2 in regulation of cellular motility of protozoan parasites, essential to invasion of host cells. Activation of CK2 or excessive activity of CK2 has been shown to occur in hosts infected with Leishmania donovani, Herpetomonas muscarum muscarum, Plasmodium falciparum, Trypanosoma brucei, Toxoplasma gondii and Schistosoma mansoni. Indeed, inhibition of CK2 has been shown to block infection by T. cruzi.

CK2 has also been shown to interact with and/or phosphorylate viral proteins associated with human immunodeficiency virus type 1 (HIV-1), human papilloma virus (HPV), and herpes simplex virus, in addition to other virus types (e.g., Epstein-Barr, human cytomegalovirus, hepatitis C and B viruses, Borna disease virus, adenovirus, coxsackievirus, coronavirus, influenza, and varicella zoster virus). CK2 phosphorylates and activates HIV-1 reverse transcriptase and proteases in vitro and in vivo, and promotes pathogenicity of simian-human immunodeficiency virus (SHIV), a model for HIV. CK2 also phosphorylates HIV-2 Nef, and it phosphorylates Vpu protein, leading to rapid loss of circulating CD4+ T-cells. Inhibitors of CK2 are thus able to reduce pathogenic effects of a model of HIV infection. CK2 also phosphorylates numerous proteins in herpes simplex virus and numerous other viruses, and some evidence suggests viruses have adopted CK2 as a phosphorylating enzyme for their essential life cycle proteins. For example, CK2 has been reported to phosphorylate E7, a viral oncoprotein upregulated in cells infected with HPVs, at the time when E7 function is optimal, i.e., when it stimulates cell cycle progression from G1 to S phase. HPVs are responsible for a number of diseases, including cervical cancer. The viral oncoproteins E6 and E7 are believed to be primarily responsible for inducing the transformed phenotype in HPV infected cells. Thus, CK2 may be effective to reduce the pathogenic effects of HPV infection by blocking phosphorylation of E7.

CK2 has also been shown to function as a key regulator of temperature compensation of circadian clocks. By controlling expression, the level of CK2 was shown to determine the form of compensation through the phosphorylation of the clock protein FREQUENCY (FRQ), which was found to be compromised in CK2 hypomorphs. By contrast, other kinases and phosphatases implicated in clock function do not play appreciable roles in temperature compensation. See Mehra et al., Cell, 2009, 137(4):749-60.

CK2 is unusual in the diversity of biological processes that it affects, and it differs from most kinases in other ways as well: it is constitutively active, it can use ATP or GTP, and it is elevated in most tumors and rapidly proliferating tissues. It also has unusual structural features that may distinguish it from most kinases, too, enabling its inhibitors to be highly specific for CK2 while many kinase inhibitors affect multiple kinases, increasing the likelihood of off-target effects, or variability between individual subjects. For all of these reasons, CK2 is a particularly interesting target for drug development, and the invention provides highly effective inhibitors of CK2 that are useful in treating a variety of different diseases and disorders mediated by or associated with excessive, aberrant or undesired levels of CK2 activity.

### DISCLOSURE OF THE INVENTION

The present invention in part provides chemical compounds having certain biological activities that include, but are not limited to, inhibiting cell proliferation, inhibiting angiogenesis, and modulating protein kinase activities. These molecules modulate casein kinase 2 (CK2) activities, and thus affect biological functions that include but are not limited to, inhibiting gamma phosphate transfer from ATP to a protein or peptide substrate, inhibiting angiogenesis, inhibiting cell proliferation and inducing cell apoptosis, for example. The present invention also in part provides methods for preparing novel chemical compounds, and analogs thereof, and methods of using these compounds. Also provided are pharmaceutical compositions comprising the above-described molecules and a pharmaceutically acceptable excipient or diluent, and methods for using such compounds and compositions. The present invention also provides compositions comprising the above-described molecules in combination with other materials, including other therapeutic agents.

In one aspect, the invention provides a method for treating or ameliorating a disorder other than a solid tumor, that is associated with undesired activity of protein kinase CK2, or a condition where inhibition of CK2 is therapeutically beneficial, which method comprises administering to a subject in need of such treatment or amelioration a therapeutically effective amount of a compound of Formula I:

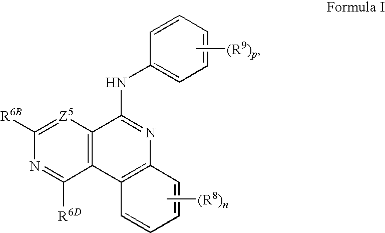

or a pharmaceutically acceptable salt or ester thereof,

wherein Z⁵ is N or CR^{6A};

each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOK, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl, and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S;

n is 0 to 4; and

p is 0 to 4.

In another aspect, the invention provides a method for treating or ameliorating a disorder associated with excessive or undesired CK2 activity other than a solid tumor, which method comprises administering to a subject in need of such treatment or amelioration a therapeutically effective amount of a compound of Formula I:

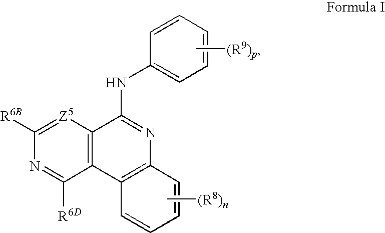

or a pharmaceutically acceptable salt or ester thereof, as described above,

wherein the disorder is selected from the group consisting of a neurodegenerative disorder, an inflammatory disorder, pain, a disorder of the vascular system, a pathophysiological disorder of skeletal muscle or bone tissue, protozoan parasitosis, a viral disease, leukemia, lymphoma, and multiple myeloma.

In a further aspect, the invention provides a method for treating or ameliorating such disorders in a subject, which method comprises administering to said subject in need of such treatment or amelioration a compound of Formula I:

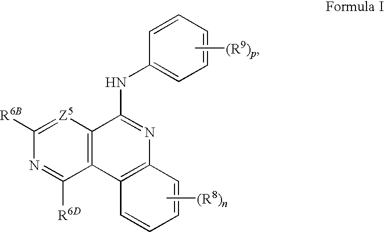

or a pharmaceutically acceptable salt or ester thereof, as described above,

in an amount effective to inhibit undesired activity of protein kinase CK2.

In a further aspect, the invention provides a method for treating or ameliorating a solid tumor, in particular an advanced solid tumor, which method comprises administering to a subject in need of such treatment or amelioration a therapeutically effective amount of a compound of Formula I:

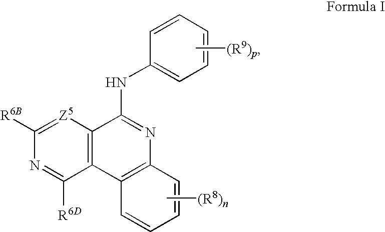

or a pharmaceutically acceptable salt or ester thereof, as further described herein.

In yet another aspect, the invention provides a method for treating, ameliorating or preventing a circadian rhythm disorder in a subject in need thereof, comprising administering to said subject a therapeutically effective amount of a compound of formula (I):

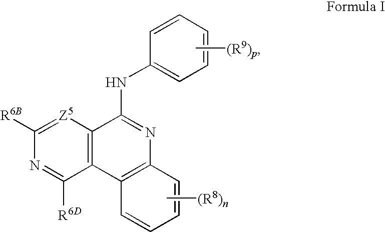

or a pharmaceutically acceptable salt or ester thereof, as further described herein.

In still another aspect, the invention provides a method for modulating temperature compensation and/or circadian rhythm, which method comprises administering to a subject in need of such modulation a therapeutically effective amount of a compound of formula (I):

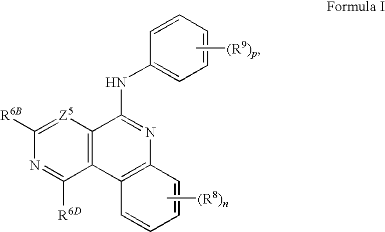

or a pharmaceutically acceptable salt or ester thereof, as further described herein.

In some embodiments, the compound of Formula I has the structure of Formula II, III, IV, V or VI,

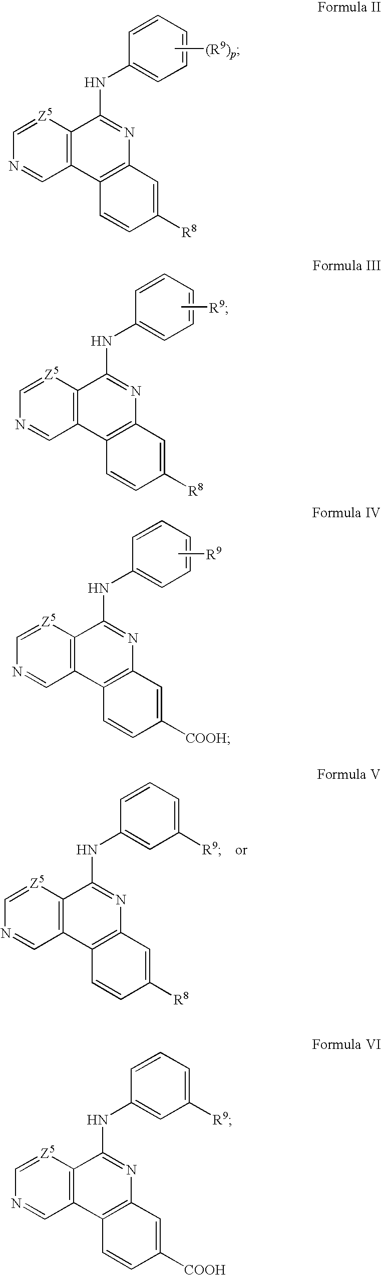

or a pharmaceutically acceptable salt or ester thereof;

wherein Z⁵ is N or CR^{6A};

each R^{6A} and R⁸ independently is H or an optionally substituted C1-C8 alkyl, C2-C8 heteroalkyl, C₂-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or each R^{6A} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S; and

p is 0 to 4.

Compounds particularly useful in the claimed methods include compounds of formula VIa:

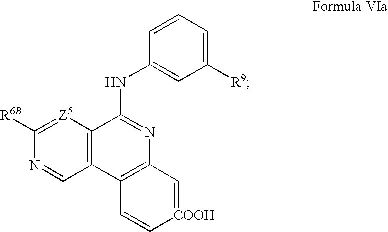

wherein R^{6B} can be H or —NHR′, where R′ is C1-C5 hydrocarbyl group, preferably C1-C3 alkyl or C3-C5 cycloalkyl; Z⁵ is CH or N; and R⁹ is halo, CF₃, or CCR″, where R″ is H or Me, and the pharmaceutically acceptable salts thereof.

In preferred embodiments of the compounds of Formula VIa, R^{6B} is H or —NH-cyclopropyl; and R⁹ is C1, CF₃, or CCH. Z⁵ is preferably CH, and R^{6B} is then preferably H. When Z⁵ is N, R^{6B} can be H or —NHR′. Compounds of Formula VIa include compounds K, (1) and (2), described below. Esters of the free carboxylic acid of compounds of Formula VIa are also included, particularly the methyl, ethyl, 2-hydroxyethyl, and 2-methoxyethyl esters.

In certain preferred embodiments, the compound of Formula I is a compound (Compound K) having the formula:

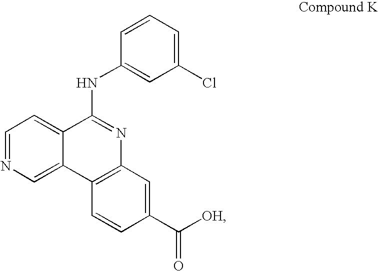

or a pharmaceutically acceptable salt or ester thereof.

In other preferred embodiments, the compound of Formula I is a compound (Compound 1 or Compound 2) having the formula:

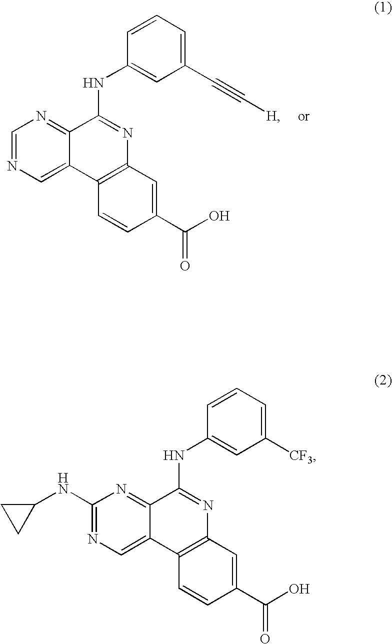

or a pharmaceutically acceptable salt or ester thereof.

The present invention provides methods for treating or ameliorating a disorder associated with undesired activity of protein kinase CK2, which method comprises administering to a subject in need of such treatment or amelioration a therapeutically effective amount of a compound (Compound K) having the formula:

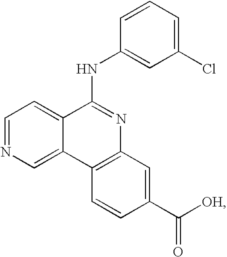

or a pharmaceutically acceptable salt or ester thereof.

In some such embodiments, the disorder to be treated or ameliorated is a disorder other than a solid tumor. In other embodiments, the disorder is a solid tumor, in particular an advanced solid tumor.

In some embodiments, the methods described herein comprise administering an effective amount of a pharmaceutical composition comprising the compound of Formula I, or a pharmaceutically acceptable salt or ester thereof, admixed with at least one pharmaceutically acceptable excipient. In some such embodiments, the pharmaceutical composition comprises a compound of Formula II, III, IV, V or VI. In other such embodiments, the pharmaceutical composition comprises a compound of Formula VIa, such as compound K, Compound (1) or Compound (2).

In some embodiments, the compound or a pharmaceutical composition comprising a compound, such as compound K, compound (1) or compound (2), is administered orally, either in solid form or as a liquid composition comprising an effective amount of the compound. Alternatively, the compound or pharmaceutical composition may be administered by injection. An effective amount can be determined by conventional methods, but is typically between 1 and 200 mg/kg. Oral dosage forms may be administered as a fixed dosage containing about 25 mg or 50 mg or 100 mg of the compound, or as a weight-adjusted dosage of the compound.

### BRIEF DESCRIPTION OF THE DRAWINGS

FIG. 1 shows that compound K causes suppression of IL-8 production in prostate cancer cells, using qRT-PCR (8 hr) (A) and ELISA (24 hr) (B).

FIG. 2 shows the effect of Compound K in a formalin-induced pain response test designed to identify a compound that provides pain relief and distinguish whether the compound has anti-inflammatory activity. It demonstrates that Compound K is effective to reduce pain responses, and indicates Compound K has anti-inflammatory activity. The first vertical bar in each graph is a control with no compound; the second bar is 30 mg/kg Compound K; the third vertical bar is 100 mg/kg Compound K; and the darker bar on the end is for 200 mg/kg Compound K. The designations * and ** indicate a statistically significant reduction in flinching. The reductions were not statistically significant in Phase I, the first 9 minutes following treatment with Compound K (A), but statistically significant effects were seen in two of the three test groups during Phase II, (B).

FIG. 3 shows the effect of Compound (1) in a formalin-induced pain response test designed to identify a compound that provides pain relief and distinguish whether the compound has anti-inflammatory activity. It demonstrates that Compound (1) is effective to reduce pain responses, and indicates Compound (1) has anti-inflammatory activity. The first vertical bar in each graph is a control with no compound; the second bar is 30 mg/kg Compound (1); the third vertical bar is 100 mg/kg Compound (1); and the darker bar on the end is for 200 mg/kg Compound (1). The designations * and ** indicate a statistically significant reduction in flinching. The reductions were not statistically significant in Phase I, the first 9 minutes following treatment with Compound (1) (A), but statistically significant effects were seen in two of the three test groups during Phase II, (B).

FIG. 4 shows the plasma concentration mean profiles of Compound K in plasma at day 1 (A) and day 21 (B) for Cohorts 1-3 of Example 5.

FIG. 5 shows inhibition of HIV-1 93TH073 Clade E replication in eight CCR5-tropic HIV-1 clinical isolates in fresh human PBMCs treated with Compound 2 (A) or AZT (B).

### MODES OF CARRYING OUT THE INVENTION

The present invention relates to compounds that inhibit CK2, and exert a variety of beneficial therapeutic effects, and provides methods to use highly potent CK2 inhibitors for treatment of various disorders.

The present invention may be understood more readily by reference to the following detailed description of the preferred embodiments of the invention and the Examples included herein. It is to be understood that unless specifically defined herein, the terminology used herein is to be given its traditional meaning as known in the relevant art.

Compounds of Formulae I, II, III, IV, V, and VI are inhibitors of CK2; compounds K, (1) and (2) are preferred examples of CK2 inhibitors for the purposes of the present invention. These compounds exert biological activities that include, but are not limited to, inhibiting cell proliferation and modulating protein kinase activity. Compounds of these Formulae can modulate protein kinase CK2 activity, and without being bound by theory, it is believed their inhibition of CK2 provides the ability to treat various disorders described herein, which are associated with aberrant, excessive, or undesired levels of CK2 activity. Such compounds therefore can be utilized in multiple applications by a person of ordinary skill in the art. For example, compounds described herein may find uses that include, but are not limited to, (i) modulation of protein kinase activity (e.g., CK2 activity), (ii) modulation of cell proliferation, (iii) modulation of apoptosis, (iv) treatment of cell proliferation related disorders, such as leukemia, lymphoma, multiple myeloma, and solid tumors, and (v) treatment of neurodegenerative disorders, inflammatory disorders, disorders of the vascular system, disorders of skeletal muscle or bone tissue, protozoan parasitosis, viral diseases, and pain.

As used herein, the singular forms “a”, “an”, and “the” include plural references unless indicated otherwise.

As used herein, the term “subject” refers to a human or animal subject. In preferred embodiments, the subject is human.

The terms “treat”, “treating” or “treatment” in reference to a particular disease or disorder includes prevention of the disease or disorder, and/or lessening, improving, ameliorating or removing the symptoms and/or pathology of the disease or disorder.

The term “therapeutically effective amount” or “effective amount” is intended to mean that amount of a drug or pharmaceutical agent that will elicit a biological or medical response of a cell, tissue, system, animal or human that is being sought by a researcher, veterinarian, medical doctor or other clinician.

A candidate molecule or compound described herein may be in a therapeutically effective amount in a pharmaceutical formulation or medicament, which is an amount that can lead to a desired biological effect, leading to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganism include but are not limited to virus, protozoa, bacterium and fungus.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-C10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain 1-10C (alkyl) or 2-10C (alkenyl or alkynyl). Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, CCR, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′ SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

“Acetylene” substituents are 2-10C alkynyl groups that are optionally substituted, and are of the formula —C≡C—R^{a}, wherein W is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and each R^{a} group is optionally substituted with one or more substituents selected from halo, ═O, ═N—CN, ═N—OR′, ═NR′, OR′, NR′₂, SR′, SO₂R′, SO₂NR′₂, NR′ SO₂R′, NR′CONR′₂, NR′COOR′, NR′COR′, CN, COOR′, CONR′₂, OOCR′, COR′, and NO₂, wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S. In some embodiments, R^{a} of —C≡C—R^{a} is H or Me.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, CCR, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ—where n is 1-8 and preferably n is 1-4, though where specified, an alkylene can also be substituted by other groups, and can be of other lengths, and the open valences need not be at opposite ends of a chain. Thus —CH(Me)— and —C(Me)₂— may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Halo”, as used herein includes fluoro, chloro, bromo and iodo. Fluoro and chloro are often preferred.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems.

As used herein, the term “heteroatom” refers to any atom that is not carbon or hydrogen, such as nitrogen, oxygen or sulfur.

Illustrative examples of heterocycles include but are not limited to tetrahydrofuran, 1,3 dioxolane, 2,3 dihydrofuran, pyran, tetrahydropyran, benzofuran, isobenzofuran, 1,3-dihydro-isobenzofuran, isoxazole, 4,5 dihydroisoxazole, piperidine, pyrrolidine, pyrrolidin-2-one, pyrrole, pyridine, pyrimidine, octahydro pyrrolo[3,4b]pyridine, piperazine, pyrazine, morpholine, thiomorpholine, imidazole, imidazolidine-2,4-dione, 1,3-dihydrobenzimidazol-2-one, indole, thiazole, benzothiazole, thiadiazole, thiophene, tetrahydro thiophene 1,1-dioxide, diazepine, triazole, guanidine, diazabicyclo[2.2.1]heptane, 2,5 diazabicyclo[2.2.1]heptane, 2,3,4,4a,9,9a hexahydro 1H β carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, azido, cyano, sulfonyls, sulfinyls, sulfonates, phosphates, etc.

The term “polar substituent” as used herein refers to any substituent having an electric dipole, and optionally a dipole moment (e.g., an asymmetrical polar substituent has a dipole moment and a symmetrical polar substituent does not have a dipole moment). Polar substituents include substituents that accept or donate a hydrogen bond, and groups that would carry at least a partial positive or negative charge in aqueous solution at physiological pH levels. In certain embodiments, a polar substituent is one that can accept or donate electrons in a non-covalent hydrogen bond with another chemical moiety. In certain embodiments, a polar substituent is selected from a carboxy, a carboxy bioisostere or other acid-derived moiety that exists predominately as an anion at a pH of about 7 to 8. Other polar substituents include, but are not limited to, groups containing an OH or NH, an ether oxygen, an amine nitrogen, an oxidized sulfur or nitrogen, a carbonyl, a nitrile, and a nitrogen-containing or oxygen-containing heterocyclic ring whether aromatic or non-aromatic. In some embodiments, the polar substituent represented by R⁸ is a carboxylate or a carboxylate bioisostere.

“Carboxylate bioisostere” or “carboxy bioisostere” as used herein refers to a moiety that is expected to be negatively charged to a substantial degree at physiological pH. In certain embodiments, the carboxylate bioisostere is a moiety selected from the group consisting of:

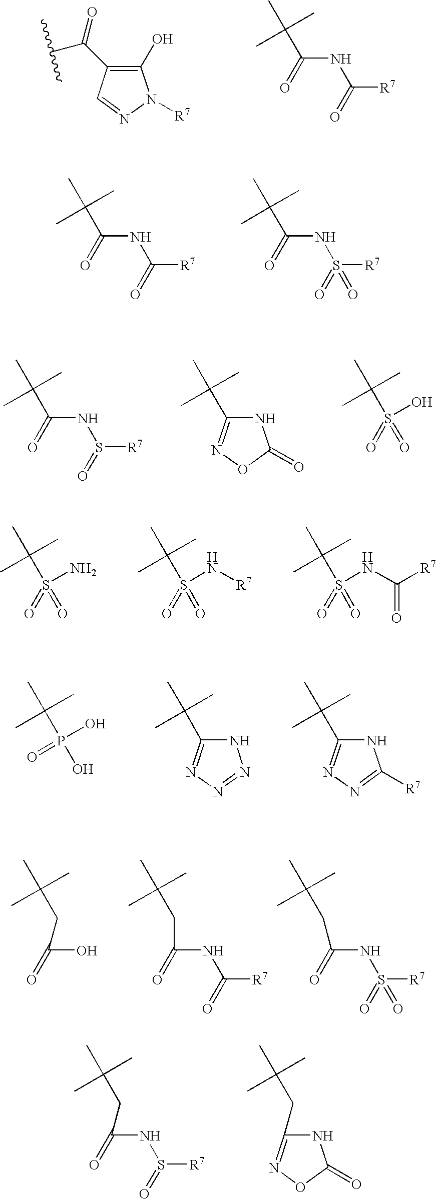

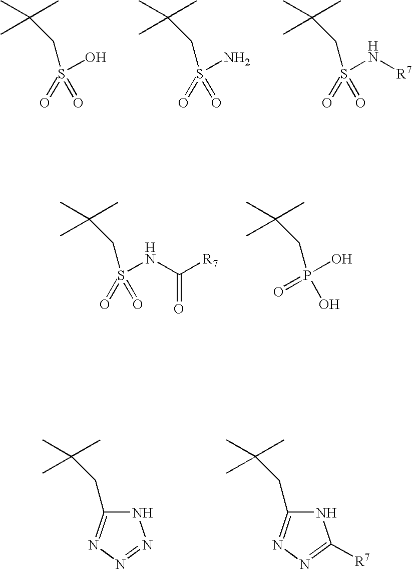

and salts and prodrugs of the foregoing, wherein each R⁷ is independently H or an optionally substituted member selected from the group consisting of C₁₋₁₀ alkyl, C₂₋₁₀ alkenyl, C₂₋₁₀ heteroalkyl, C₃₋₈ carbocyclic ring, and C₃₋₈ heterocyclic ring optionally fused to an additional optionally substituted carbocyclic or heterocyclic ring; or le is a C₁₋₁₀ alkyl, C₂₋₁₀ alkenyl, or C₂₋₁₀ heteroalkyl substituted with an optionally substituted C₃₋₈ carbocyclic ring or C₃₋₈ heterocyclic ring.

In certain embodiments, the polar substituent is selected from the group consisting of carboxylic acid, carboxylic ester, carboxamide, tetrazole, triazole, carboxymethanesulfonamide, oxadiazole, oxothiadiazole, thiadiazole, thiazole, aminothiazole and hydroxythiazole.

In some embodiments, at least one R⁸ present is a carboxylic acid or a salt, or ester or a bioisostere thereof. In certain embodiments, at least one R⁸ present is a carboxylic acid-containing substituent or a salt, ester or bioisostere thereof. In the latter embodiments, the R⁸ substituent may be a C1-C10 alkyl or C1-C10 alkenyl linked to a carboxylic acid (or salt, ester or bioisostere thereof).

In one aspect, the present invention provides a method for treating or ameliorating a disorder associated with undesired activity of protein kinase CK2, which method comprises administering to a subject in need of such treatment or amelioration a therapeutically effective amount of a compound of Formula I, II, III, IV, V, or VI, as described herein:

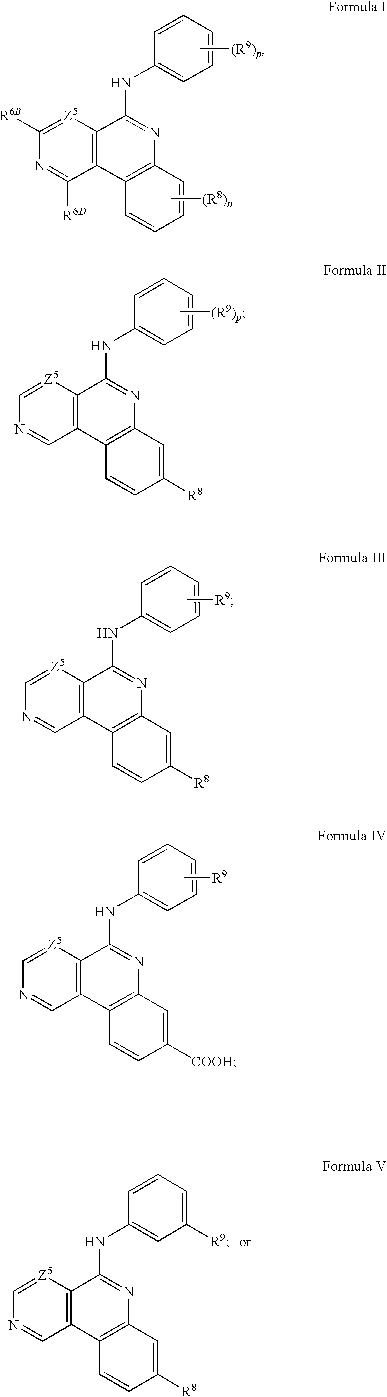

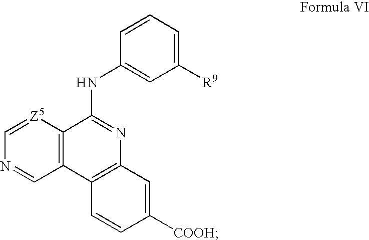

or a pharmaceutically acceptable salt or ester thereof.

In particular, compounds of Formula VIa are of interest for the methods of the invention:

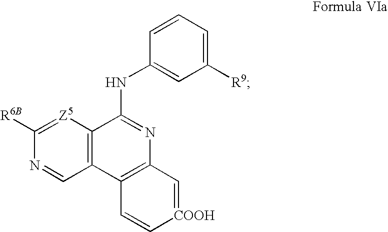

wherein R^{6B} can be H or —NHR′, where R′ is C1C5 hydrocarbyl group, preferably C1-C3 alkyl or C3-C5 cycloalkyl; Z⁵ is CH or N; and R⁹ is halo, CF₃, or CCR″, where R″ is H or Me.

In preferred embodiments of the compounds of Formula VIa, R^{6B} is H or —NH-cyclopropyl; and R⁹ is C1, CF₃, or CεCH. Z⁵ is preferably CH, and R^{6B} is then preferably H. When Z⁵ is N, R^{6B} can be H or —NHR′. Compounds of Formula VIa include compounds K, (1) and (2), described below. Esters of the free carboxylic acid of compounds of Formula VIa are also included, particularly the methyl, ethyl, 2-hydroxyethyl, and 2-methoxyethyl esters.

In some such embodiments, the disorder is a neurodegenerative disorder, an inflammatory disorder, a disorder of the vascular system, a pathophysiological disorder of skeletal muscle or bone tissue, protozoan parasitosis, a viral disease, leukemia, lymphoma, multiple myeloma, a solid tumor, including an advanced solid tumor, or Castleman's disease.

In another aspect, the invention provides a method for treating or ameliorating a disorder in a subject, which method comprises administering to said subject in need of such treatment or amelioration a therapeutically effective amount of a compound of Formula I, II, III, IV, V, or VI, as described herein, or a pharmaceutically acceptable salt or ester thereof, wherein the disorder is selected from the group consisting of a neurodegenerative disorder, an inflammatory disorder, pain, a disorder of the vascular system, a pathophysiological disorder of skeletal muscle or bone tissue, protozoan parasitosis, a viral disease, leukemia, lymphoma, multiple myeloma, a solid tumor, including an advanced solid tumor, or Castleman's disease.

In another aspect, the invention provides a method for treating or ameliorating a disorder in a subject, which method comprises administering to said subject in need of such treatment or amelioration a compound of Formula I, II, III, IV, V, or VI, as described herein, or a pharmaceutically acceptable salt or ester thereof, in an amount effective to inhibit undesired activity of protein kinase CK2.

In some such embodiments, the disorder is a neurodegenerative disorder, an inflammatory disorder, pain, a disorder of the vascular system, a pathophysiological disorder of skeletal muscle or bone tissue, protozoan parasitosis, a viral disease, leukemia, lymphoma, multiple myeloma, a solid tumor, including an advanced solid tumor, or Castleman's disease.

In certain embodiments, the disorder to be treated or ameliorated by the methods described herein is a neurodegenerative disorder. In some such embodiments, the neurodegenerative disorder is Alzheimer's disease, Parkinson's disease, memory impairment, brain ischemia, Guam-Parkinson dementia, chromosome 18 deletion syndrome, progressive supranuclear palsy, Kuf's disease, or Pick's disease.

In other embodiments, the disorder to be treated or ameliorated by the methods described herein is an inflammatory disorder. Sometimes, the inflammatory disorder is glomerulonephritis, multiple sclerosis, systemic lupus erythematosus, rheumatoid arthritis, or juvenile arthritis In some embodiments, the compounds are used to alleviate inflammatory pain, since murine models demonstrate that CK2 modulates nociceptive signal transmission, and reduces pain response in mice when infused into the spinal cord.

Compounds of the invention were shown to be effective for treatment of pain associated with inflammation in a formalin-induced pain model. In particular, the tested compounds were active during the second phase of testing by the formalin-induced method described by Hunskaar, et al. (“The formalin test in mice: Dissociation between inflammatory and non-inflammatory pain,” Pain 30, 103-14 (1987).) Hunskaar describes a two-phase test, observing responses to formalin injection into the paw of a mouse. Reduction in pain response during the first few minutes after injection of formalin indicates a general antinociceptive response characteristic of centrally-acting analgesics that lack anti-inflammatory activity, e.g. morphine. Compounds like naproxen and steroids that act as anti-inflammatory agents affect only the later phase in this test. The compounds of the invention behave as anti-inflammatory agents in this test, thus the compounds disclosed herein are useful to treat pain, including acute or chronic inflammatory pain, and pain that persists for a period of an hour or more (persistent pain). Because they act during the second phase of this test rather than both phases, they are indicated to have anti-inflammatory activity and are useful to treat inflammation as well as pain associated with inflammation.

CK2 has also been shown to play a role in the pathogenesis of atherosclerosis, and may prevent atherogenesis by maintaining laminar shear stress flow. CK2 plays a role in vascularization, and has been shown to mediate the hypoxia-induced activation of histone deacetylases (HDACs). CK2 is also involved in diseases relating to skeletal muscle and bone tissue, including, e.g., cardiomyocyte hypertrophy, heart failure, impaired insulin signaling and insulin resistance, hypophosphatemia and inadequate bone matrix mineralization.

Thus in one aspect, the invention provides methods to treat each of these conditions, comprising administering to a subject in need of such treatment an effect amount of a CK2 inhibitor, such as a compound of Formula I-VI or VIa, as described herein.

In further embodiments, the disorder to be treated or ameliorated by the methods described herein is a disorder of the vascular system. In some such embodiments, the disorder of the vascular system is atherosclerosis, laminar shear stress or hypoxia.

The invention also in part pertains to methods for modulating an immune response in a subject, and methods for treating a condition associated with an aberrant immune response in a subject. Thus, provided are methods for determining whether a compound herein modulates an immune response, which comprise contacting a system with a compound described herein in an amount effective for modulating (e.g., inhibiting) an immune response or a signal associated with an immune response. Signals associated with immunomodulatory activity include, e.g., stimulation of T-cell proliferation, suppression or induction of cytokines, including, e.g., interleukins, interferon-γ and TNF. Methods of assessing immunomodulatory activity are known in the art.

Also provided are methods for treating a condition associated with an aberrant immune response in a subject, which comprise administering a compound described herein to a subject in need thereof in an amount effective to treat the condition. Conditions characterized by an aberrant immune response include without limitation, organ transplant rejection, asthma, autoimmune disorders, including rheumatoid arthritis, multiple sclerosis, myasthenia gravis, systemic lupus erythematosus, scleroderma, polymyositis, mixed connective tissue disease (MCTD), Crohn's disease, and ulcerative colitis. In certain embodiments, an immune response may be modulated by administering a compound herein in combination with a molecule that modulates (e.g., inhibits) the biological activity of an mTOR pathway member or member of a related pathway (e.g., mTOR, PI3 kinase, AKT). In certain embodiments the molecule that modulates the biological activity of an mTOR pathway member or member of a related pathway is rapamycin. In certain embodiments, provided herein is a composition comprising a compound described herein in combination with a molecule that modulates the biological activity of an mTOR pathway member or member of a related pathway, such as rapamycin, for example.

In other embodiments, the disorder to be treated or ameliorated by the methods described herein is a pathophysiological disorder of skeletal muscle or bone tissue. These conditions include atherosclerosis, laminar shear stress, and hypoxia and associated conditions. In some such embodiments, the disorder to be treated by the methods of the invention is cardiomyocyte hypertrophy, impaired insulin signaling or bone tissue mineralization.

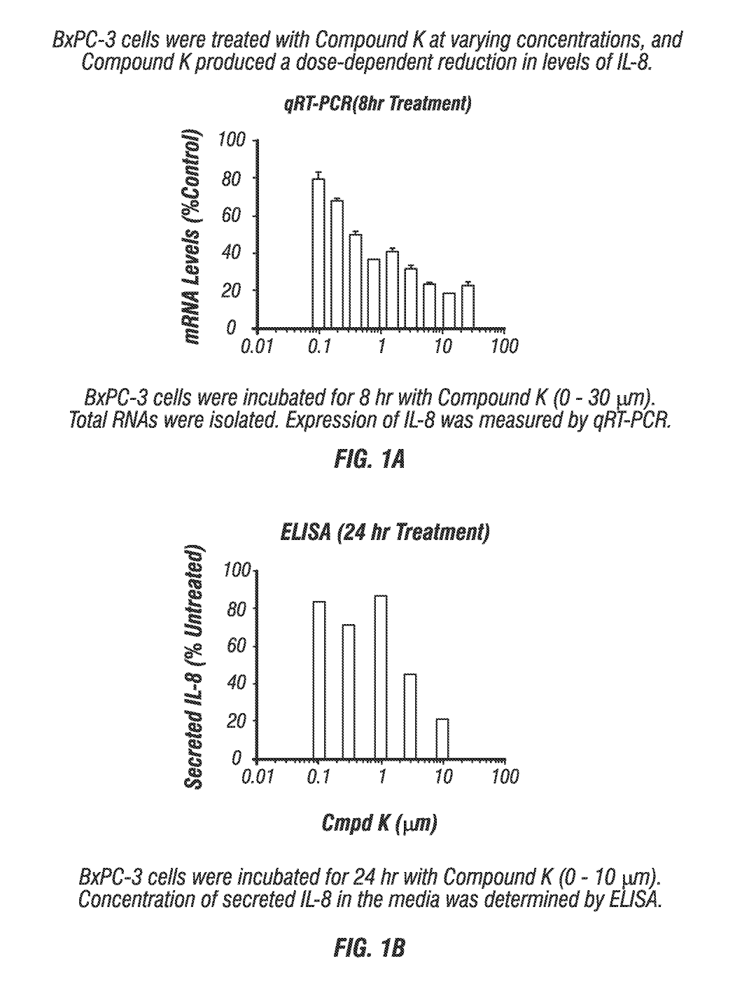

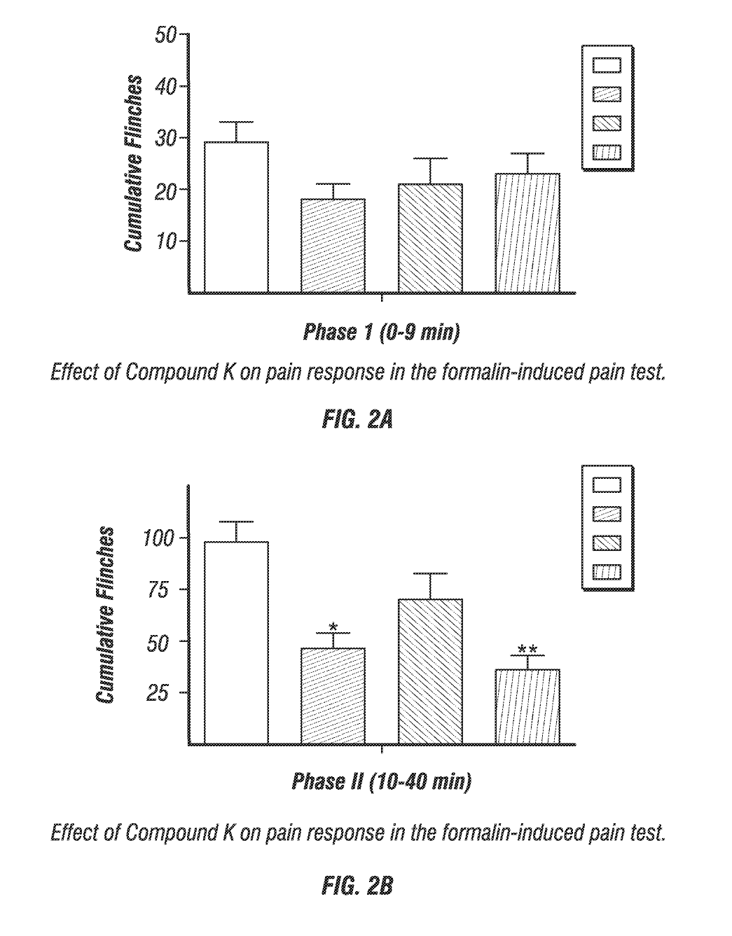

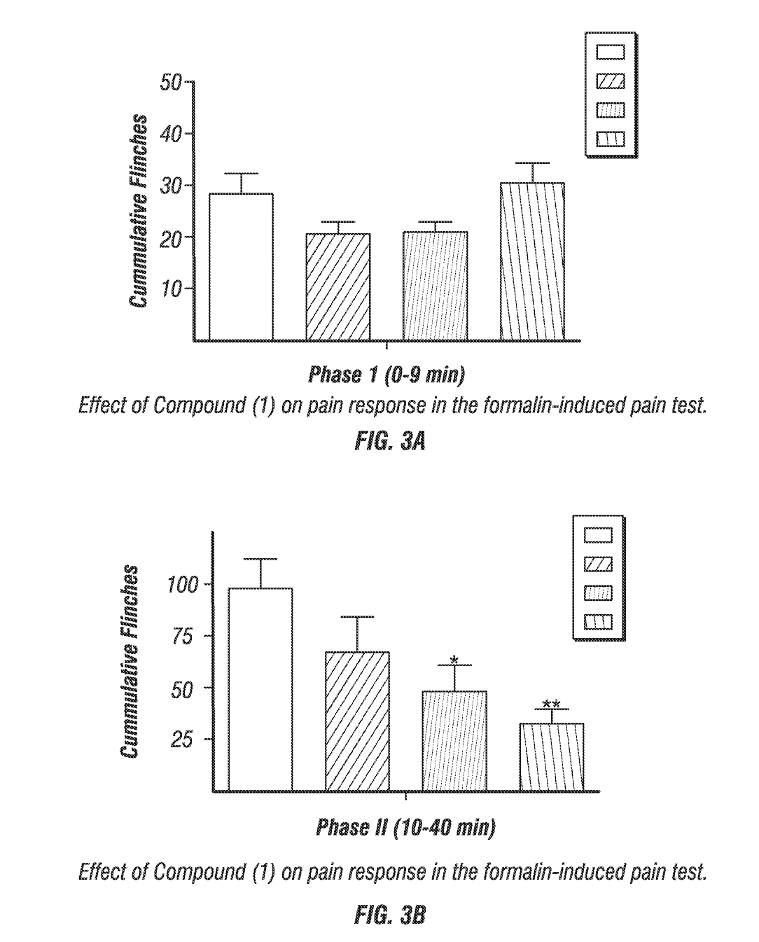

In still other embodiments, the disorder to be treated or ameliorated by the methods described herein is a protozoan parasitosis. Infections by protozoans have been shown to lead to almost immediate increases in IL-8 levels in the infected host. It has now been shown that treatment with compounds of Formula VIa suppresses secretion of IL-8. See FIG. 1. In addition to the involvement of CK2 inhibitors in the life cycle of such pathogens, which is discussed above, the suppression of IL-8 expression may be helpful in ameliorating localized injury associated with parasitic pathogens. The compounds of the invention are thus useful for treatment of parasitosis due to Theileria parva; Toxoplasma gondii, Trypanosoma cruzi (Chagas disease), Leishmania donovani, Herpetomonas muscarum muscarum, Plasmodium falciparum, Traypanosoma brucei, and Schistosoma mansoni, among others.

In further embodiments, the disorder to be treated or ameliorated is a viral disease. In some such embodiments, the viral disease is human immunodeficiency virus type 1 (HIV-1), human papilloma virus, Epstein-Barr virus or herpes simplex virus. In other embodiments, the viral disorder is human papilloma virus, human cytomegalovirus, hepatitis C or B, Borna disease virus, adenovirus, coxsackie virus, coronavirus, or varicella zoster virus.

In still other embodiments, the disorder to be treated or ameliorated by the methods described is leukemia (e.g., acute myelogenous leukemia, chronic myelogenous leukemia, ALL, and Bcr/Abl-positive leukemia), lymphoma, or multiple myeloma. In other embodiments, the disorder is a solid tumor. In some embodiments, the solid tumor is an advanced solid tumor. Sometimes, the solid tumor is a squamous cell carcinoma, or an adenocarcinoma of the colon, rectum, kidney, breast or prostate. In other embodiments, the compounds are used to treat pancreatic cancer, hepatocellular carcinoma, neuroblastoma, or rhabdomyosarcoma tumors (RMS). For treatment of RMS, the compounds can be used in combination with a TRAIL receptor ligand. In other embodiments, the disorder is Castelman's disease.

The invention also provides methods for treating, ameliorating or preventing a circadian rhythm disorder in a subject in need thereof, comprising administering to said subject a therapeutically effective amount of a CK2 inhibitor, such as a compound of Formula I-VI or VIa, as described herein. In some embodiments, the circadian rhythm disorder is selected from jet lag, shift work sleep disorder, and sleep disorders, including, e.g., delayed sleep phase syndrome (DSPS), advanced sleep phase syndrome, and non 24-hour sleep wake disorder. In other embodiments, the circadian rhythm disorder is winter depression or seasonal affective disorder.

The invention further provides methods for modulating temperature compensation and/or circadian rhythm, which method comprises administering to a subject in need of such modulation a therapeutically effective amount of a CK2 inhibitor, such as a compound of Formula I-VI or VIa, as described herein.

In some embodiments, a compound identified herein is useful to reset the circadian clock. The compound can be used to treat or prevent jet lag or facilitate resetting the clock in shift workers, or to treat, ameliorate or prevent sleep disorders, including, e.g., delayed sleep phase syndrome (DSPS), advanced sleep phase syndrome, and non 24-hour sleep wake disorder. In addition, the compound can be used to improve rhythmicity, i.e., the coordinated regulation of outputs from cells within the suprachiasmatic nucleus (SCN). Disruption of rhythmicity is common in the elderly and affects the ability to sleep. The compounds described herein can be used to improve the interactions between neurons to allow them to arrive at a common phase or directly reset individual neurons to a common phase. Compounds can also be used to alleviate circadian rhythm disorders such as winter depression or seasonal affective disorder.

In certain preferred embodiments of the methods described herein, the compound of Formula (I) is Compound K:

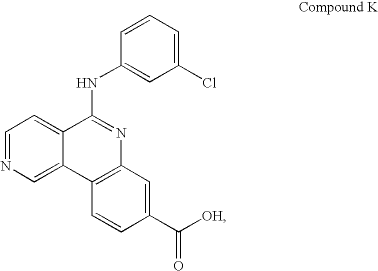

or a pharmaceutically acceptable salt or ester thereof.

Compound K is a highly potent and selective inhibitor of CK2. Its IC₅₀ against CK2 is about 2 nM. It was tested for activity against a battery of about 145 kinases, and the highest activity found (lowest IC₅₀) was on CK2. Other kinases where it showed the most activity are shown in the following table (Table 1). On other kinases, the IC₅₀ was higher, so Compound K is selective for CK2.

TABLE 1

| Kinase | IC-50 (nM) |
|---|---|
| CK2α | 1 |
| CK2α′ | 3 |
| DAPK3 | 17 |
| FLT3 | 35 |
| HIPK3 | 45 |
| PIM1 | 46 |
| Cdk1/Cycl B | 56 |
| DYRK2 | 91 |

In other preferred embodiments, the compound of Formula I is a compound having the formula of Compound 1 or Compound 2:

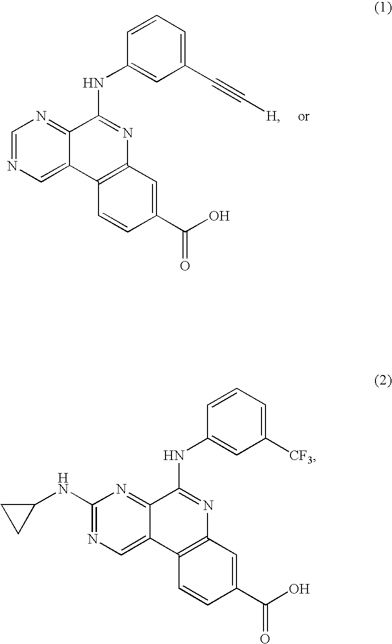

or a pharmaceutically acceptable salt or ester thereof.

Compound 1 exhibited an IC₅₀ of 6 nM for inhibition of CK2. Compound 2 exhibited an IC₅₀ of about 9 nM for inhibition of CK2.

In preferred embodiments of the methods described herein, the subject is human. Typically the subject is one who has been diagnosed as in need of treatment for one or more of the conditions described herein; the methods of the invention optionally include identifying a suitable subject for treatment. The methods further optionally include determining a level of CK2 in the subject or in an appropriate tissue from the subject, such as in a tissue affected by the disorder to be treated. In some embodiments, progress or effectiveness of a treatment method disclosed herein can be monitored by determining whether the level of CK2 or of CK2 activity in the subject, or in the tissue affected by the disorder, is reduced by treatment with a compound in accordance with the invention.

### Formulation and Administration

While the compositions and methods of the present invention will typically be used in therapy for human patients, they may also be used in veterinary medicine to treat similar or identical diseases. The compositions may, for example, be used to treat mammals, including, but not limited to, primates and domesticated mammals. The compositions may, for example be used to treat herbivores. The compositions of the present invention include geometric and optical isomers of one or more of the drugs, wherein each drug is a racemic mixture of isomers or one or more purified isomers.

Pharmaceutical compositions suitable for use in the present invention include compositions wherein the active ingredients are contained in an effective amount to achieve the intended purpose. Determination of the effective amounts is well within the capability of those skilled in the art, especially in light of the detailed disclosure provided herein.

The compounds of the present invention may exist as pharmaceutically acceptable salts. The present invention includes such salts. The term “pharmaceutically acceptable salts” is meant to include salts of active compounds which are prepared with relatively nontoxic acids or bases, depending on the particular substituent moieties found on the compounds described herein. When compounds of the present invention contain relatively acidic functionalities, base addition salts can be obtained by contacting the neutral form of such compounds with a sufficient amount of the desired base, either neat or in a suitable inert solvent. Included are base addition salts such as sodium, potassium, calcium, ammonium, organic amino, or magnesium salt, or a similar salt. In some embodiments, the compounds such as compounds K, (1), or (2) are administered as a sodium salt, and may be administered in either a solid form or in a liquid form.

When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids, for example, acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate, glutamate, and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof, including racemic mixtures), succinates, benzoates and salts with amino acids such as glutamic acid. These salts may be prepared by methods known to those skilled in art.

The neutral forms of the compounds are conveniently regenerated by contacting the salt with a base or acid and isolating the parent compound in the conventional manner. The parent form of the compound differs from the various salt forms in certain physical properties, such as solubility in polar solvents.

The pharmaceutically acceptable esters in the present invention refer to non-toxic esters, preferably the alkyl esters such as methyl, ethyl, propyl, isopropyl, butyl, isobutyl or pentyl esters, of which the ethyl or methyl esters are preferred. However, other esters such as phenyl-C₁₋₅ alkyl may be employed if desired. Ester derivatives of certain compounds may act as prodrugs which, when absorbed into the bloodstream of a warm-blooded animal, may cleave in such a manner as to release the drug form and permit the drug to afford improved therapeutic efficacy.

Certain compounds of the present invention are isolated as solids and can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. Therefore, single stereochemical isomers as well as enantiomeric and diastereomeric mixtures of the present compounds are within the scope of the invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another. It will be apparent to one skilled in the art that certain compounds of this invention may exist in tautomeric forms, all such tautomeric forms of the compounds being within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention. The compounds of the present invention may also contain unnatural proportions of atomic isotopes at one or more of atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the present invention, whether radioactive or not, are encompassed within the scope of the present invention.

In addition to salt forms, the present invention provides compounds that are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

When used as a therapeutic, the compounds described herein often are administered with a physiologically acceptable carrier. A physiologically acceptable carrier is a formulation to which the compound can be added to dissolve it or otherwise facilitate its administration. Examples of physiologically acceptable carriers include, but are not limited to, water, saline, phosphate buffer, or physiologically buffered saline.

A compound of the present invention can be formulated as a pharmaceutical composition. Such a pharmaceutical composition can then be administered orally, parenterally, by inhalation spray, rectally, or topically in dosage unit formulations containing conventional nontoxic pharmaceutically acceptable carriers, adjuvants, and vehicles as desired. Topical administration can also involve the use of transdermal administration such, as transdermal patches or iontophoresis devices. The term parenteral as used herein includes subcutaneous injections, intravenous, intramuscular, intrasternal injection, or infusion techniques. Formulation of drugs is discussed in, for example, Hoover, John E., REMINGTON′S PHARMACEUTICAL SCIENCES, Mack Publishing Co., Easton, Pa.; 1975. Other examples of drug formulations can be found in Liberman, H. A. and Lachman, L., Eds., PHARMACEUTICAL DOSAGE FORMS, Marcel Decker, New York, N.Y., 1980.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

Suppositories for rectal administration of the drug can be prepared by mixing the drug with a suitable nonirritating excipient such as cocoa butter, synthetic mono-di- or triglycerides, fatty acids and polyethylene glycols that are sold at ordinary temperatures but liquid at the rectal temperature and will therefore melt in the rectum and release the drug.

Solid dosage forms for oral administration can include capsules, tablets, pills, powders, and granules. In such solid dosage forms, the compounds of this invention are ordinarily combined with one or more adjuvants appropriate to the indicated route of administration. If administered per os, a compound of the invention can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. A compound of the invention can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

In some embodiments, a compound or it sodium salt is administered in the form of a solid tablet or pill, admixed with inert carriers; in other embodiments, the compound or its sodium salt is dissolved or suspended in a liquid medium such as phosphate-buffered saline, and solubility is facilitated by addition of a small amount of a polyethylene glycol, such as PEG-300. These formulations can be administered orally, or the liquid formulation can be prepared and delivered by injection. In some embodiments, the compounds of the invention, such as compound K, or (1), or (2), is formulated in a PEG-containing buffered solution.

In some embodiments, the compound or a pharmaceutical composition comprising a compound, such as compound K, compound (1) or compound (2), is administered orally, either in solid form or as a liquid composition comprising an effective amount of the compound. Alternatively, it may be administered by injection. An effective amount can be determined by conventional methods, but is typically between 1 and 200 mg/kg. Oral dosage forms may be administered as a fixed dosage containing about 25 mg or 50 mg or 100 mg of the compound, or as a weight-adjusted dosage of the compound.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage form varies depending upon the mammalian host treated and the particular mode of administration. An effective amount can be determined by conventional methods, but is typically between 1 and 200 mg/kg. Oral dosage forms may be administered as a fixed dosage containing about 25 mg or 50 mg or 100 mg of the compound, or as a weight-adjusted dosage of the compound, such as 30 mg/kg or 60 mg/kg, or 100-200 mg/kg. Dosages may be administered at any appropriate frequency, but in some embodiments because of the plasma half-life of the compounds, the methods include administration either once per day or twice per day. Dosing may continue at the judgment of the treating physician for any appropriate time, but in some embodiments a compound of the invention will be administered once or twice per day for about a week or about two weeks.

The dosage regimen utilizing the compounds of the present invention, alone or in combination with an anticancer agent, is selected in accordance with a variety of factors including type, species, age, weight, sex and medical condition of the patient; the severity of the condition to be treated; the route of administration; the renal and hepatic function of the patient; and the particular compound or salt or ester thereof employed. A consideration of these factors is within the purview of the ordinarily skilled clinician for the purpose of determining the therapeutically effective dosage amounts to be given to a person in need of the instant therapy or combination therapy.

While various routes of administration can be used for the compounds and methods of the invention, in some embodiments the methods use compound K or a salt thereof for oral administration, to treat one or more of the conditions described herein. Compound K exhibits good oral bioavailability in mouse, rat and dog testing (ca. 20-50%) when administered orally, in the form of filled gelatin capsules. It exhibits a half-life between about 5 and 12 hours across this range of test subjects, and a relatively low clearance rate in vivo, thus its overall oral bioavailability is good. Based on the pharmacokinetic profile and half-life information, in some embodiments, compound K would be administered orally, twice per day, to provide efficacious plasma levels. Particularly for chronic conditions, oral administration is preferred. Typical dosages for oral administration would be approximately 5-500 mg/kg per day.

As demonstrated by Example 3, compounds of the invention can be used orally to reduce pain. For treatment of pain, the compounds can be administered orally or by injection methods. For treatment of persistent pain, compounds may be administered orally one or two times per day, or more than twice per day; in some embodiments, a compound of the invention is administered once or twice per day, at a weight-normalized daily dosage of about 1-500 mg/kg, preferably 10-300 mg/kg. Each dose can be administered at 10-300 mg/kg once per day in some embodiments, or at a dose of about 10-200 mg/kg twice per day in some embodiments. When two daily doses are administered, they are often administered at least four hours apart, preferably at least about 8 hours apart.

In some embodiments, the methods for treating pain use a fixed dosage such as a specific tablet or capsule size rather than being normalized to the subject's body weight. In such embodiments, each dose can be between 10 and 500 mg, and is often between 20 and 300 mg.

Oral administration for treatment of pain can use a solid formulation, or a liquid formulation. The liquid formulation can be a solution or suspension. Both solid and liquid formulations can be formulated as described herein, using one or more carriers and/or pharmaceutically acceptable excipients.

For some embodiments, delivery of compound K by injection, such as intramuscular or intravenous, may be preferred. When injected, the half-life for compound K is still about 5-12 hours, which is virtually the same as for oral delivery. The dosage for injected delivery (IV) can be about half of the oral dosage since injection bypasses the absorption barriers that reduce oral bioavailability. For delivery by injection, lower doses such as 1-25 mg/kg per day may be used to achieve similar plasma levels of drug.

Therapeutic Combinations

Compounds of the invention may be used alone or in combination with another therapeutic agent. The invention provides methods to treat conditions such as cancer, inflammation and immune disorders by administering to a subject in need of such treatment a therapeutically effective amount of a therapeutic agent useful for treating said disorder and administering to the same subject a a therapeutically effective amount of a modulator of the present invention. A CK2 modulator is an agent that inhibits or enhances a biological activity of a CK2 protein and is generically referred to hereafter as a “modulator.”

Compounds of Formula I are exemplary ‘modulators.’ The therapeutic agent and the modulator may be administered together, either as separate pharmaceutical compositions or admixed in a single pharmaceutical composition. The therapeutic agent and the modulator may also be administered separately, including at different times and with different frequencies. The modulator may be administered by any known route, such as orally, intravenously, intramuscularly, nasally, and the like; and the therapeutic agent may also be administered by any conventional route. In many embodiments, at least one and optionally both of the modulator and the therapeutic agent may be administered orally. Preferably, the modulator is a CK2 inhibitor, and provides the treatment effects described herein.

In certain embodiments, a “modulator” as described above may be used in combination with a therapeutic agent that can act by binding to regions of DNA that can form certain quadruplex structures. In such embodiments, the therapeutic agents have anticancer activity on their own, but their activity is enhanced when they are used in combination with a modulator. This synergistic effect allows the therapeutic agent to be administered in a lower dosage while achieving equivalent or higher levels of at least one desired effect.

The amount of each of these materials to be administered will vary with the route of administration, the condition of the subject, other treatments being administered to the subject, and other parameters. The therapeutic agents of the invention may, of course, cause multiple desired effects; and the amount of modulator to be used in combination with the therapeutic agent should be an amount that increases one or more of these desired effects. The modulator is to be administered in an amount that is effective to enhance a desired effect of the therapeutic agent. An amount is “effective to enhance a desired effect of the therapeutic agent”, as used herein, if it increases by at least about 25% at least one of the desired effects of the therapeutic agent alone. Preferably, it is an amount that increases a desired effect of the therapeutic agent by at least 50% or by at least 100% (i.e., it doubles the effective activity of the therapeutic agent.) In some embodiments, it is an amount that increases a desired effect of the therapeutic agent by at least 200%.

The amount of a modulator that increases a desired effect of a therapeutic agent may be determined using in vitro methods, such as cell proliferation assays. The therapeutic agents of the invention are useful to counter hyperproliferative disorders such as cancer, thus they reduce cell proliferation. Thus, for example, a suitable amount of a modulator could be the amount needed to enhance an antiproliferative effect of a therapeutic agent by at least 25% as determined in a cell proliferation assay.

The modulator used in the present invention may enhance at least one desired effect produced by the therapeutic agent it is used with, thus the combinations of the invention provide a synergistic effect, not merely an additive effect. The modulators themselves are at times useful for treating the same types of conditons, and thus may also have some direct effect in such assays. In that event, the “amount effective to increase a desired effect” must be a synergistic enhancement of the activity of the therapeutic agent that is attributable to enhancement by the modulator of an effect of the therapeutic agent, rather than a simple additive effect that would be expected with separate administration of the two materials. In many cases, the modulator can be used in an amount (concentration) that would not be expected to have any apparent effect on the treated subject or the in vitro assay, so the increased effect achieved with the combination is directly attributable to a synergistic effect.

For administration to animal or human subjects, the appropriate dosage of a modulator, such as a compound of Formula I, II, III, IV, V or VI as described herein, is typically between about 0.01-15 mg/kg, and about 0.1-10 mg/kg. Dosage levels are dependent on the nature of the condition, drug efficacy, the condition of the patient, the judgment of the practitioner, and the frequency and mode of administration; however, optimization of such parameters is within the ordinary level of skill in the art.

A modulator may be separately active for treating a cancer. For combination therapies described above, when used in combination with a therapeutic agent, the dosage of a modulator will frequently be two-fold to ten-fold lower than the dosage required when the modulator is used alone to treat the same condition or subject. Determination of a suitable amount of the modulator for use in combination with a therapeutic agent is readily determined by methods known in the art.

Compounds and compositions of the invention may be used in combination with anticancer or other agents, such as palliative agents, that are typically administered to a patient being treated for cancer. Such “anticancer agents” include, e.g., classic chemotherapeutic agents, as well as molecular targeted therapeutic agents, biologic therapy agents, and radiotherapeutic agents.

When a compound or composition of the invention is used in combination with an anticancer agent or another therapeutic agent, the present invention provides, for example, simultaneous, staggered, or alternating treatment. Thus, the compound of the invention may be administered at the same time as an anticancer or additional therapeutic agent, in the same pharmaceutical composition; the compound of the invention may be administered at the same time as the other agent, in separate pharmaceutical compositions; the compound of the invention may be administered before the other agent, or the other agent may be administered before the compound of the invention, for example, with a time difference of seconds, minutes, hours, days, or weeks.

In examples of a staggered treatment, a course of therapy with the compound of the invention may be administered, followed by a course of therapy with another therapeutic agent, or the reverse order of treatment may be used, and more than one series of treatments with each component may also be used. In certain examples of the present invention, one component, for example, the compound of the invention or the other therapeutic agent, is administered to a mammal while the other component, or its derivative products, remains in the bloodstream of the mammal. For example, a compound for formulae (I)-(VI) may be administered while the other agent or its derivative products remains in the bloodstream, or the other therapeutic agent may be administered while the compound of formulae (I)-(VI) or its derivatives remains in the bloodstream. In other examples, the second component is administered after all, or most of the first component, or its derivatives, have left the bloodstream of the mammal.

The compound of the invention and the additional therapeutic agent may be administered in the same dosage form, e.g., both administered as intravenous solutions, or they may be administered in different dosage forms, e.g., one compound may be administered topically and the other orally. A person of ordinary skill in the art would be able to discern which combinations of agents would be useful based on the particular characteristics of the drugs and the cancer involved.

Additional therapeutic agents useful for therapy in combination with the compounds of the invention include the following types of agents and inhibitors:

Anticancer agents useful in combination with the compounds of the present invention may include agents selected from any of the classes known to those of ordinary skill in the art, including, but not limited to, antimicrotubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; nonreceptor tyrosine kinase angiogenesis inhibitors; immunotherapeutic agents; pro-apoptotic agents; and cell cycle signaling inhibitors; other agents.

Anti-microtubule or anti-mitotic agents are phase specific agents that are typically active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that are believed to operate at the G2/M phases of the cell cycle. It is believed that the diterpenoids stabilize the p-tubulin subunit of the microtubules, by binding with this protein. Disassembly of the protein appears then to be inhibited with mitosis being arrested and cell death following.

Examples of diterpenoids include, but are not limited to, taxanes such as paclitaxel, docetaxel, larotaxel, ortataxel, and tesetaxel. Paclitaxel is a natural diterpene product isolated from the Pacific yew tree Taxus brevifolia and is commercially available as an injectable solution TAXOL®. Docetaxel is a semisynthetic derivative of paclitaxel q. v., prepared using a natural precursor, 10-deacetyl-baccatin III, extracted from the needle of the European Yew tree. Docetaxel is commercially available as an injectable solution as TAXOTERE®.

Vinca alkaloids are phase specific anti-neoplastic agents derived from the periwinkle plant. Vinca alkaloids that are believed to act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, vindesine, and vinorelbine. Vinblastine, vincaleukoblastine sulfate, is commercially available as VELBAN® as an injectable solution. Vincristine, vincaleukoblastine 22-oxo-sulfate, is commercially available as ONCOVIN® as an injectable solution. Vinorelbine, is commercially available as an injectable solution of vinorelbine tartrate (NAVELBINE®), and is a semisynthetic vinca alkaloid derivative.

Platinum coordination complexes are non-phase specific anti-cancer agents, which are interactive with DNA. The platinum complexes are believed to enter tumor cells, undergo, aquation and form intra- and interstrand crosslinks with DNA causing adverse biological effects to the tumor. Platinum-based coordination complexes include, but are not limited to cisplatin, carboplatin, nedaplatin, oxaliplatin, satraplatin, and (SP-4-3)-(cis)-amminedichloro[2-methylpyridine]platinum(II). Cisplatin, cis-diamminedichloroplatinum, is commercially available as PLATINOL® as an injectable solution. Carboplatin, platinum, diammine [1,1-cyclobutane-dicarboxylate(2-)-0,0′], is commercially available as PARAPLATIN® as an injectable solution.

Alkylating agents are generally non-phase specific agents and typically are strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, alkyl sulfonates such as busulfan; ethyleneimine and methylmelamine derivatives such as altretamine and thiotepa; nitrogen mustards such as chlorambucil, cyclophosphamide, estramustine, ifosfamide, mechlorethamine, melphalan, and uramustine; nitrosoureas such as carmustine, lomustine, and streptozocin; triazenes and imidazotetrazines such as dacarbazine, procarbazine, temozolamide, and temozolomide. Cyclophosphamide, 2-[bis(2-chloroethyl)-amino]tetrahydro-2H-1,3,2-oxazaphosphorine 2-oxide monohydrate, is commercially available as an injectable solution or tablets as CYTOXAN®. Melphalan, 4-[bis(2-chloroethyl)amino]-L-phenylalanine, is commercially available as an injectable solution or tablets as ALKERAN®. Chlorambucil, 4-[bis(2-chloroethyl)amino]-benzenebutanoic acid, is commercially available as LEUKERAN® tablets. Busulfan, 1,4-butanediol dimethanesulfonate, is commercially available as MYLERAN® TABLETS. Carmustine, 1,3-[bis(2-chloroethyl)-1-nitrosourea, is commercially available as single vials of lyophilized material as BiCNU®., 5-(3,3-dimethyl-1-triazeno)-imidazole-4-carboxamide, is commercially available as single vials of material as DTIC-Dome®.

Anti-tumor antibiotics are non-phase specific agents which are believed to bind or intercalate with DNA. This may result in stable DNA complexes or strand breakage, which disrupts ordinary function of the nucleic acids, leading to cell death. Examples of anti-tumor antibiotic agents include, but are not limited to, anthracyclines such as daunorubicin (including liposomal daunorubicin), doxorubicin (including liposomal doxorubicin), epirubicin, idarubicin, and valrubicin; streptomyces-related agents such as bleomycin, actinomycin, mithramycin, mitomycin, porfiromycin; and mitoxantrone. Dactinomycin, also know as Actinomycin D, is commercially available in injectable form as COSMEGEN®. Daunorubicin, (8S-cis-)-8-acetyl-10-10-[(3-amino-2,3,6-trideoxy-α-L-lyxohexopyranosyl)oxy]-7,8,9,10-tetrahydro-6,8,11-trihydroxy-1-methoxy-5,12-naphthacenedione hydrochloride, is commercially available as a liposomal injectable form as DAUNOXOME® or as an injectable as CERUBIDINE®. Doxorubicin, (8S,10S)-10-[(3-amino-2,3,6-trideoxy-a-L-lyxohexopyranosyl)oxy]-8-glycoloyl, 7,8,9,10-tetrahydro-6,8,11-trihydroxy-1-methoxy-5,12-naphthacenedione hydrochloride, is commercially available in an injectable form as RUBEX® or ADRIAMYCIN RDF®. Bleomycin, a mixture of cytotoxic glycopeptide antibiotics isolated from a strain of Streptomyces verticillus, is commercially available as BLENOXANE®.

Topoisomerase II inhibitors include, but are not limited to, epipodophyllotoxins, which are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide, teniposide, and amsacrine. Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Teniposide, 4′-demethyl-epipodophyllotoxin 9[4,6-0-(R)-thenylidene-β-D-glucopyranoside], is commercially available as an injectable solution as VUMON® and is commonly known as VM-26.

Antimetabolite neoplastic agents are phase specific anti-neoplastic agents that typically act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Anti-metabolites, include purine analogs, such as fludarabine, cladribine, chlorodeoxyadenosine, clofarabine, mercaptopurine, pentostatin, erythrohydroxynonyladenine, fludarabine phosphate and thioguanine; pyrimidine analogs such as fluorouracil, gemcitabine, capecitabine, cytarabine, azacitidine, edatrexate, floxuridine, and troxacitabine; antifolates, such as methotrexate, pemetrexed, raltitrexed, and trimetrexate. Cytarabine, 4-amino-1-p-D-arabinofuranosyl-2(1H)-pyrimidinone, is commercially available as CYTOSAR-U® and is commonly known as Ara-C. Mercaptopurine, 1,7-dihydro-6H-purine-6-thione monohydrate, is commercially available as PURINETHOL®. Thioguanine, 2-amino-1,7-dihydro-6H-purine-6-thione, is commercially available as TABLOID®. Gemcitabine, 2′-deoxy-2′,2′-difluorocytidine monohydrochloride (p-isomer), is commercially available as GEMZAR®.

Topoisomerase I inhibitors including, camptothecin and camptothecin derivatives. Examples of topoisomerase I inhibitors include, but are not limited to camptothecin, topotecan, irinotecan, rubitecan, belotecan and the various optical forms (i.e., (R), (S) or (R,S)) of 7-(4-methylpiperazino-methylene)-10,11-ethylenedioxy-camptothecin, as described in U.S. Pat. Nos. 6,063,923; 5,342,947; 5,559,235; 5,491,237 and pending U.S. patent application Ser. No. 08/977,217 filed Nov. 24, 1997. Irinotecan HCl, (4S)-4,11-diethyl-4-hydroxy-9-[(4-piperidinopiperidino)-carbonyloxy]-1H-pyrano[3′,4′,6,7]indolizinol-1,2-biquinoline-3, 14(4H,12H)-dione hydrochloride, is commercially available as the injectable solution CAMPTOSAR®. Irinotecan is a derivative of camptothecin which binds, along with its active metabolite 8N-38, to the topoisomerase I-DNA complex. Topotecan HCl, (S)-10-[dimethylamino]methyl′-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, androgens such as fluoxymesterone and testolactone; antiandrogens such as bicalutamide, cyproterone, flutamide, and nilutamide; aromatase inhibitors such as aminoglutethimide, anastrozole, exemestane, formestane, vorazole, and letrozole; corticosteroids such as dexamethasone, prednisone and prednisolone; estrogens such as diethylstilbestrol; antiestrogens such as fulvestrant, raloxifene, tamoxifen, toremifene, droloxifene, and iodoxyfene, as well as selective estrogen receptor modulators (SERMS) such those described in U.S. Pat. Nos. 5,681,835, 5,877,219, and 6,207,716; 5α-reductases such as finasteride and dutasteride; gonadotropin-releasing hormone (GnRH) and analogues thereof which stimulate the release of leutinizing hormone (LH) and/or follicle stimulating hormone (FSH), for example LHRH agonists and antagonists such as buserelin, goserelin, leuprolide, and triptorelin; progestins such as medroxyprogesterone acetate and megestrol acetate; and thyroid hormones such as levothyroxine and liothyronine.

Signal transduction pathway inhibitors are those inhibitors, which block or inhibit a chemical process which evokes an intracellular change, such as cell proliferation or differentiation. Signal tranduction inhibitors useful in the present invention include, e.g., inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases. Receptor tyrosine kinases are transmembrane proteins having an extracellular ligand binding domain, a transmembrane domain, and a tyrosine kinase domain Receptor tyrosine kinases are involved in the regulation of cell growth and are sometimes termed growth factor receptors.

Inappropriate or uncontrolled activation of many of these kinases, for example by over-expression or mutation, has been shown to result in uncontrolled cell growth. Accordingly, the aberrant activity of such kinases has been linked to malignant tissue growth. Consequently, inhibitors of such kinases could provide cancer treatment methods.

Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene.

Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al., Drug Discov. Today (1997), 2(2):50-63; and Lofts, F. J. et al., “Growth factor receptors as targets”, New Molecular Targets for Cancer Chemotherapy, ed. Workman, Paul and Kerr, David, CRC press 1994, London. Specific examples of receptor tyrosine kinase inhibitors include, but are not limited to, sunitinib, erlotinib, gefitinib, and imatinib.

Tyrosine kinases which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., J. Hematotherapy & Stem Cell Res. (1999) 8(5): 465-80; and Bolen, J. B., Brugge, J. S., Annual Review of Immunology. (1997) 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E., J. Pharmacol. Toxicol. Methods. (1995), 34(3): 125-32 Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., J. Biochemistry. (1999) 126 (5): 799-803; Brodt, P, Samani, A, & Navab, R, Biochem. Pharmacol. (2000) 60:1101-1107; Massague, J., Weis-Garcia, F., Cancer Surv. (1996) 27:41-64; Philip, P. A, and Harris, A L, Cancer Treat. Res. (1995) 78: 3-27; Lackey, K. et al. Bioorg. Med. Chem. Letters, (2000) 10(3): 223-226; U.S. Pat. No. 6,268,391; and Martinez-Lacaci, I., et al., Int. J. Cancer (2000), 88(1): 44-52 Inhibitors of Phosphotidyl inositol-3 Kinase family members including blockers of PI3-kinase, ATM, DNA-PK, and Ku are also useful in the present invention. Such kinases are discussed in Abraham, R T. Current Opin. Immunol. (1996), 8(3): 412-8; Canman, C. E., Lim, D. S., Oncogene (1998) 17(25): 3301-8; Jackson, S. P., Int. J. Biochem. Cell Biol. (1997) 29(7):935-8; and Zhong, H. et al., Cancer Res. (2000) 60(6):1541-5. Also useful in the present invention are Myo-inositol signaling inhibitors such as phospholipase C blockers and Myoinositol analogues. Such signal inhibitors are described in Powis, G., and Kozikowski A, (1994) NEW MOLECULAR TARGETS FOR CANCER CHEMOTHERAPY, ed., Paul Workman and David Kerr, CRC Press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R, Gervasoni, S I, Matar, P., J. Biomed. Sci. (2000) 7(4): 292-8; Ashby, M. N., Curr. Opin. Lipidol. (1998) 9(2): 99-102; and Oliff, A., Biochim. Biophys. Acta, (1999) 1423(3):C19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al., Cancer Treat. Rev., (2000) 26(4): 269-286); Herceptin® erbB2 antibody (see Stern, D F, Breast Cancer Res. (2000) 2(3):176-183); and 2CB VEGFR2 specific antibody (see Brekken, R. A. et al., Cancer Res. (2000) 60(18):5117-24).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphav beta3) that will inhibit angiogenesis; endostatin and angiostatin (non-RTK) may also prove useful in combination with the disclosed erb family inhibitors. (See Bruns, C J et al., Cancer Res. (2000), 60(11): 2926-2935; Schreiber A B, Winkler M E, & Derynck R., Science (1986) 232(4755):1250-53; Yen L. et al., Oncogene (2000) 19(31): 3460-9).

Agents used in immunotherapeutic regimens may also be useful in combination with the compounds of formula (I)-(V). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T, et al., Cancer Res. (2000) 60(13):3569-76; and Chen Y, et al., Cancer Res. (1998) 58(9):1965-71.

Agents used in pro-apoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family. Therefore, strategies designed to downregulate the expression of bcl-2 in tumors have demonstrated clinical benefit and are now in Phase II/III trials, namely Genta's G3139 bcl-2 antisense oligonucleotide. Such pro-apoptotic strategies using the antisense oligonucleotide strategy for bcl-2 are discussed in Waters J S, et al., J. Clin. Oncol. (2000) 18(9): 1812-23; and Kitada S, et al. Antisense Res. Dev. (1994) 4(2): 71-9.

Cell cycle signaling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signaling are under development. For instance, examples of cyclin dependent kinases, including CDK2, CDK4, and CDK6 and inhibitors for the same are described in, for instance, RosaniaGR & Chang Y-T., Exp. Opin. Ther. Patents (2000) 10(2):215-30.

Other molecular targeted agents include FKBP binding agents, such as the immunosuppressive macrolide antibiotic, rapamycin; gene therapy agents, antisense therapy agents, and gene expression modulators such as the retinoids and rexinoids, e.g. adapalene, bexarotene, trans-retinoic acid, 9-cisretinoic acid, and N-(4 hydroxyphenyl)retinamide; phenotype-directed therapy agents, including: monoclonal antibodies such as alemtuzumab, bevacizumab, cetuximab, ibritumomab tiuxetan, rituximab, and trastuzumab; immunotoxins such as gemtuzumab ozogamicin, radioimmunoconjugates such as 131-tositumomab; and cancer vaccines.

Miscellaneous agents include altretamine, arsenic trioxide, gallium nitrate, hydroxyurea, levamisole, mitotane, octreotide, procarbazine, suramin, thalidomide, photodynamic compounds such as methoxsalen and sodium porfimer, and proteasome inhibitors such as bortezomib.

Biologic therapy agents include: interferons such as interferon-u2a and interferon-u2b, and interleukins such as aldesleukin, denileukin diftitox, and oprelvekin.

In addition to these anticancer agents intended to act against cancer cells, combination therapies including the use of protective or adjunctive agents, including: cytoprotective agents such as armifostine, dexrazonxane, and mesna, phosphonates such as pamidronate and zoledronic acid, and stimulating factors such as epoetin, darbeopetin, filgrastim, PEG-filgrastim, and sargramostim, are also envisioned.

### Methods for Synthesizing Compounds of the Invention

Compounds of the invention can be synthesized by methods known in the art, including methods disclosed in International Patent Application No. PCT/US2007/077464. Representative synthesis methods are provided below.

### Process 1

3-bromo-4-pyridine carboxylic acid (3.0 g, 14.9 mmol) in ethanol (100 mL) was treated with concentrated sulfuric acid (5 mL).

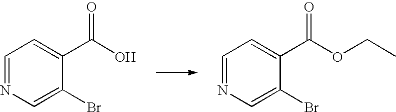

The mixture was brought to reflux at which time everything went into solution. After 12 hours at reflux, LCMS indicated that the reaction was complete. The reaction mixture was cooled to room temperature and concentrated on a rotary evaporator to a third of its original volume. The mixture was then diluted with 250 mL of ethyl acetate and washed twice with saturated aqueous sodium bicarbonate. Concentration on a rotary evaporator yielded 3.25 g of the ethyl ester as a yellowish oil which was sufficiently pure enough for subsequent chemical transformations. LCMS (ESI) 216.2 (M+1)⁺

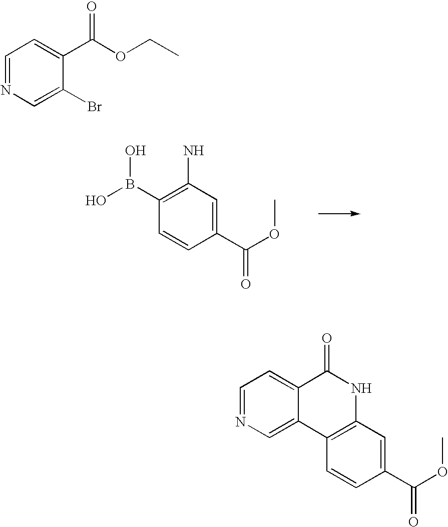

Ethyl 3-bromo-4-pyridine carboxylate 1.15 g, 5.0 mmol), 2-amino-4-methoxycarbonyl-phenylboronic acid (1.04 g, 4.5 mmol), sodium acetate (1.64 g, 20 mmol), 1,1′-bis(diphenylphosphino)ferrocene palladium (II) chloride (complexed with dichloromethane) (182 mg, 0.25 mmol) and dimethylformamide (7.5 mL) were combined in a flask. The flask was evacuated and filled with nitrogen twice and heated to 125° C. with stirring for 12 hours or until LCMS indicated the absence of any starting material. The mixture was cooled to room temperature and water (100 mL) was added to form a brown precipitate. The precipitate was filtered to yield 637 mg of methyl 5-oxo-5,6-dihydrobenzo[c][2,6]naphthyridine-8-carboxylate. LCMS (ESI) 255.4 (M+1)⁺.

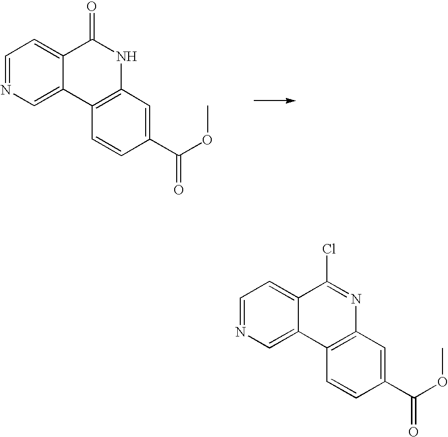

Methyl 5-oxo-5,6-dihydrobenzo[c][2,6]naphthyridine-8-carboxylate (200 mg, 0.787 mmol) was combined with phosphorus oxychloride (1 mL) and heated to reflux. After 2 hours, LCMS indicated the absence of any starting material. The volatiles were removed under reduced pressure. The residue was taken up in dichloromethane (50 mL) and washed twice with saturated aqueous sodium bicarbonate. The organic phase was dried over sodium sulfate and concentrated on a rotary evaporator to give methyl 5-chlorobenzo[c][2,6]naphthyridine-8-carboxylate (140 mg) as a grayish solid. LCMS (ESI) 273.3 (M+1)⁺.

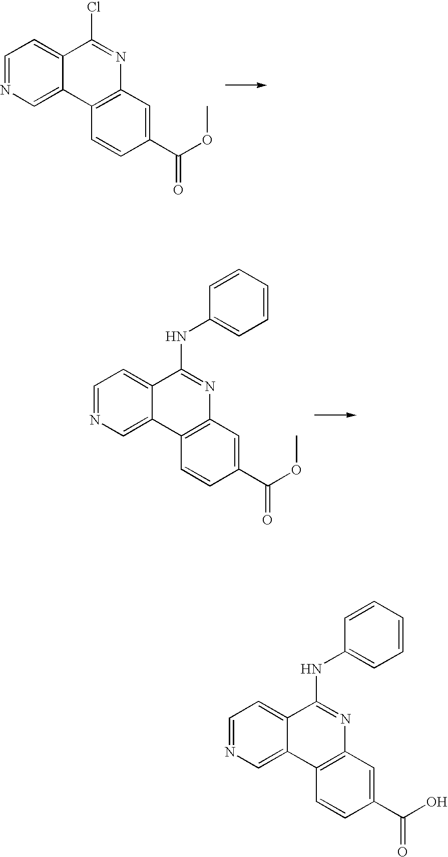

Methyl 5-chlorobenzo[c][2,6]naphthyridine-8-carboxylate (20 mg, 0.074 mmol) was combined with aniline (60 mg, 0.65 mmol) and N-methylpyrrolidinone (0.2 mL) in a microwave tube and the mixture was heated to 120° C. for 10 minutes at which time LCMS indicated that the reaction was complete as indicated by the absence of any starting material. The mixture was then purified by HPLC to yield the ester (22 mg) or it could be treated with 6N sodium hydroxide to yield the acid (19 mg). LCMS (ESI) 316.3 (M+1)⁺. ¹HNMR (400 MHz, CD₃OD) 10.17 (1H, s), 9.67 (1H, br), 8.99 (1H, d, 5.9 Hz), 8.83 (1H, d, 8.6 Hz), 8.62 (1H, d, 5.9 Hz), 8.24 (1H, d, 1.6 Hz), 8.04 (1H, s), 8.02 (1H, s), 7.93 (1H, dd, 8.2, 1.6 Hz), 7.43 (1H, d, 7.4 Hz), 7.41 (1H, d, 7.4 Hz), 7.10 (1H, m).

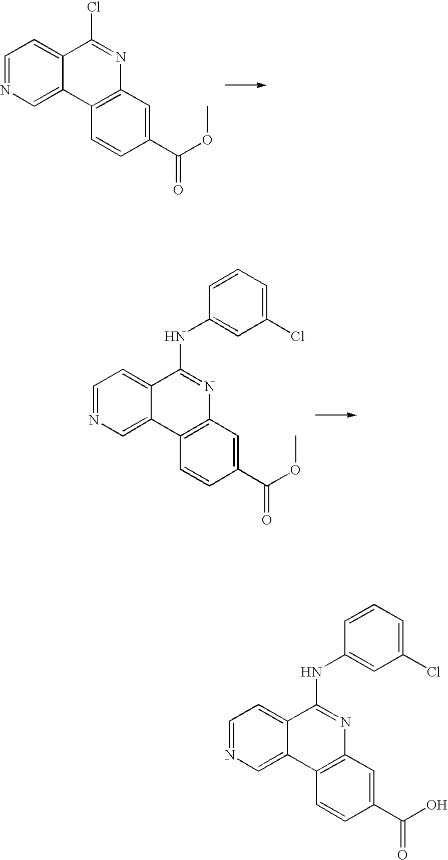

Methyl 5-chlorobenzo[c][2,6]naphthyridine-8-carboxylate (232 mg, 0.853 mmol) was combined with meta-chloroaniline (217 mg, 1.71 mmol) and N-methylpyrrolidinone (1 mL) in a flask and the mixture was heated to 80° C. for 2 hours at which time LCMS indicated that the reaction was complete as indicated by the absence of any starting material. The mixture was dissolved in CH₂Cl₂, washed with saturated aqueous sodium bicarbonate and dried over Na₂SO₄. The material was purified by flash chromatography (Sifₜ, 1:1 to 9:1 gradient of EtOAc/Hexanes) to obtain the ester. The material was dissolved in methanol and 6N aqueous NaOH and the mixture stirred at 50° C. for 30 minutes. The volatiles were removed in vacuo. The residue was triturated from acetic acid/THF/methanol using a mixture of hexanes and ethylacetate. Filtration and drying provided 147 mg of 5-(3-chlorophenylamino)benzo[c][2,6]naphthyridine-8-carboxylic acid. LCMS (ESI) 350 (M+1)⁺. ¹HNMR (400 MHz, DMSO-d₆) δ 10.21 (s, 1H), 9.72 (br s, 1H), 9.02 (d, J=5.6, 1H), 8.89 (d, J=8.8, 1H), 8.62 (d, J=5.6, 1H), 8.31 (br s, 1H), 8.28 (d, J=1.6, 1H), 8.10 (br d, J=8, 1H), 7.99 (dd, J=2, J=8.4, 1H), 7.46 (t, J=8.0, 1H), 7.16 (br d, J=7.2, 1H) ppm.

### Process 2

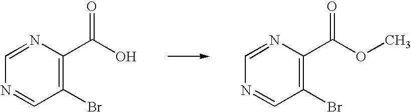

5-bromopyrimidine-4-carboxylic acid (prepared according to the procedure described in U.S. Pat. No. 4,110,450) (1.0 eq, 6.14 g, 30.2 mmol) was suspended in CH₂Cl₂ (100 ml). Oxalylchloride (1.1 eq, 2.9 ml, 33.0 mmol) was added followed by 2 drops of DMF. The mixture was stirred at room temperature overnight and the volatiles were removed in vacuo. The residue was taken in MeOH (50 ml) and heated. After evaporation of MeOH in vacuo the compound was dissolved in CH₂Cl₂ and poured on a prepacked silica gel column. The material was eluted using 20% Ethyl acetate in hexanes. Evaporation of the solvent provided methyl-5-bromopyrimidine-4-carboxylate as a light orange crystalline solid (2.54 g, 39% yield). LCMS (ES): 95% pure, m/z 217 [M]⁺; 219 [M+2]⁺; NMR (CDCl₃, 400 MHz) δ 4.04 (s, 3H), 9.02 (s, 1H), 9.21 (s, 1H) ppm.

### Process 3

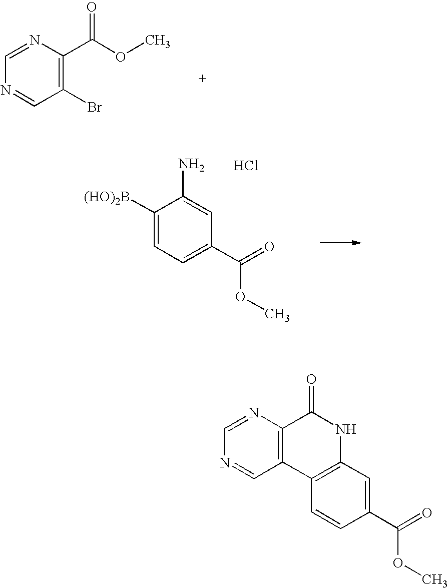

Sodium acetate (4.0 eq, 1.92 g, 23.41 mmol) and 1,1′-bis(diphenylphosphino)ferrocene palladium (II) chloride (complexed with dichloromethane) (0.05 eq, 214 mg, 0.29 mmol) were added to a mixture of methyl-5-bromopyrimidine-4-carboxylate (1.0 eq, 1.27 g, 5.85 mmol), and 2-amino-4-(methoxycarbonyl)phenylboronic acid hydrochloride (1.0 eq, 1.35 g, 5.85 mmol) in anhydrous DMF (10 ml). The Mixture was stirred under nitrogen atmosphere at 120° C. for 18 hours. Water and brine were added and the resulting solid impurities filtered off. The material was extracted with CH₂Cl₂ (4×) and the combined extracts dried over Na₂SO₄. After evaporation of CH₂Cl₂, the remaining DMF was evaporated by heating the residue in vacuo. The resulting solid was triturated in CH₂Cl₂, filtered and dried to provide methyl 5-oxo-5,6-dihydropyrimido[4,5-c]quinoline-8-carboxylate as a beige solid (127 mg, 8.5% yield). LCMS (ES): >80% pure, m/z 256 [M+1]⁺; NMR (DMSO-d₆, 400 MHz) δ 3.79 (s, 3H), 7.81 (d, J=8.0, 1H), 8.68 (d, J=8.8, 1H), 9.49 (s, 1H), 10.19 (s, 1H), 12.37 (s, 1H) ppm.

### Process 4

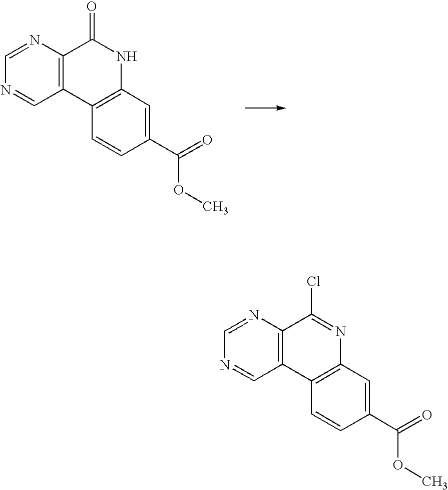

In a vial, methyl 5-oxo-5,6-dihydropyrimido[4,5-c]quinoline-8-carboxylate (1.0 eq, 151 mg, 0.59 mmol) was mixed in toluene (1 ml) with DIEA (1.5 eq, 155 ul, 0.89 mmol) and POCl₃ (5 eq, 270 ul, 3.0 mmol). The mixture was stirred at 120° C. for 1 hour and cooled down to room temperature. After adding ice and water the compound was extracted with CH₂Cl₂ (4×). The solution was filtered over Na₂SO₄ and filtered through a pad of celite. After evaporation of the volatiles, the material was triturated in a mixture of ethyl acetate and hexanes, filtered and dried to afford methyl 5-chloropyrimido[4,5-c]quinoline-8-carboxylate as a light brown fluffy solid (115 mg, 71% yield). LCMS (ES): 95% pure, m/z 274 [M+1]⁺. ¹H NMR (DMSO-d₆, 400 MHz) δ 3.96 (s, 3H), 8.37 (dd, J=1.6, J=8.4, 1H), 8.60 (d, J=1.6, 1H), 9.15 (d, J=8.8, 1H), 9.74 (s, 1H), 10.61 (s, 1H) ppm

### Process 5

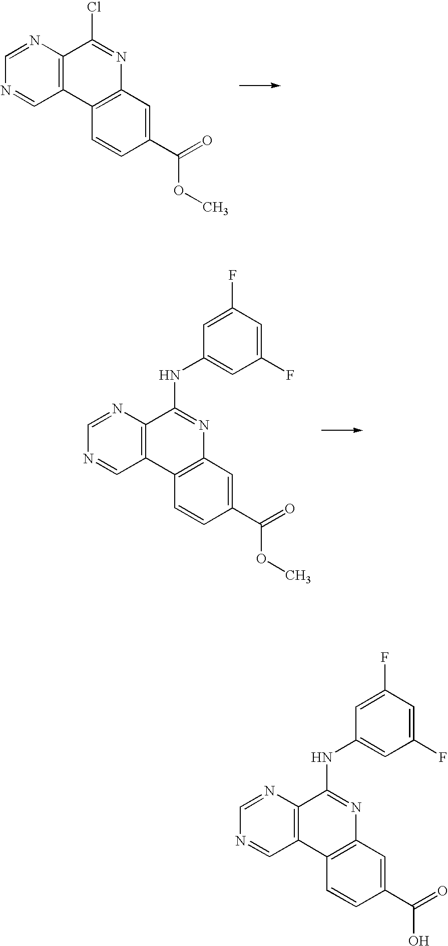

Methyl 5-chloropyrimido[4,5-c]quinoline-8-carboxylate (10 mg) was mixed with 3,5-difluoroaniline (100 mg) in NMP (0.1 ml). The mixture was heated under microwaves at 120° C. for 10 minutes. Water was added and the material extracted with CH₂Cl₂. The solvent was removed. Trituration in a mixture of ethyl acetate and hexanes and filtration provided methyl 5-(3,5-difluorophenylamino)pyrimido[4,5-c]quinoline-8-carboxylate. This material was suspended in a 1:1 mixture of THF and MeOH (2 ml) and a 5N aqueous solution of Lithium Hydroxide was added. The mixture was vigorously stirred at room temperature for 5 hours. Water and 6N hydrochloric acid were added to induce precipitation of the expected material. The solid was filtered, washed with water, dried and suspended in MeOH. Filtration and drying gave 5-(3,5-difluorophenylamino)pyrimido[4,5-c]quinoline-8-carboxylic acid as a yellow solid (4 mg, 31% yield). LCMS (ES): 95% pure, m/z 353 [M+1]⁺. ¹H NMR (DMSO-d₆, 400 MHz) δ 6.90 (br t, J=9.6, 1H), 8.02 (dd, J=1.6, J=8.0, 1H), 8.18 (br d, J=10.8, 2H), 8.34 (d, J=1.6, 1H), 8.86 (d, J=8.4, 1H), 9.65 (s, 1H), 10.40 (s, 1H), 10.44 (s, 1H) ppm.

### Process 6

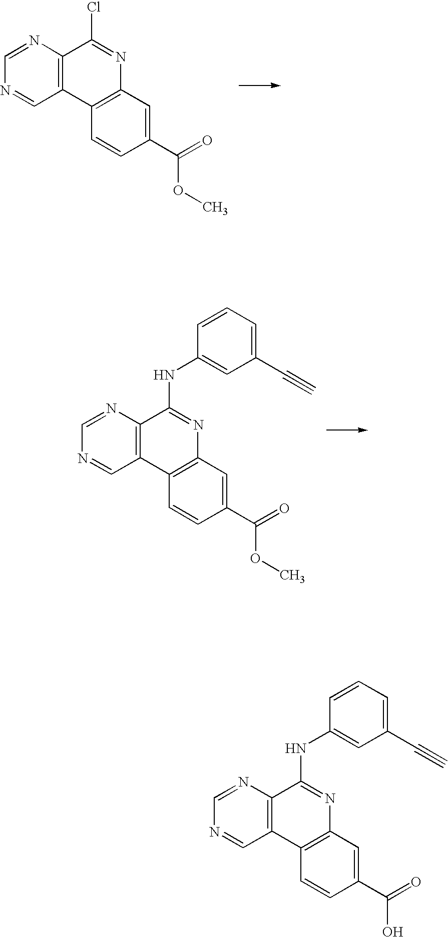

5-(3-ethynylphenylamino)pyrimido[4,5-c]quinoline-8-carboxylic acid was prepared using the same method, starting from methyl 5-chloropyrimido[4,5-c]quinoline-8-carboxylate and 3-ethynylaniline. LCMS (ES): 95% pure, m/z 341 [M+1]⁺. ¹H NMR (DMSO-d₆, 400 MHz) δ 4.20 (s, 1H), 7.19 (d, J=7.6, 1H), 7.42 (t, J=8.0, 1H), 7.99 (dd, J=1.6, J=8.4, 1H), 8.30 (d, J=1.6, 1H), 8.34 (dd, J=1.6, J=8.0, 1H), 8.49 (br s, 1H), 8.85 (d, J=8.8, 1H), 9.65 (s, 1H), 10.11 (s, 1H), 10.43 (s, 1H) ppm.

### Process 7

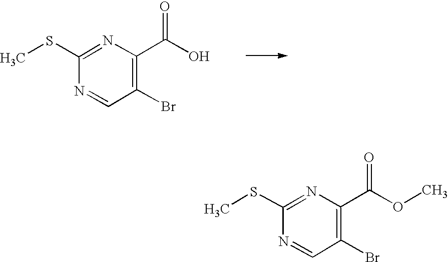

methyl-5-bromo-2-(methylthio)pyrimidine-4-carboxylate was prepared according to the procedure used in process 2 for the preparation of methyl-5-bromopyrimidine-4-carboxylate. LCMS (ES): >90% pure, m/z 263 [M]⁺, 265 [M+2]⁺; ¹H NMR (CDCl₃, 400 MHz) δ 2.59 (s, 3H), 4.00 (s, 3H), 8.71 (s, 1H) ppm.

### Process 8

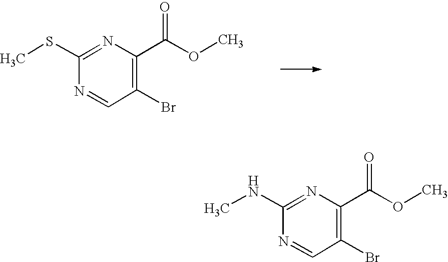

Methyl-5-bromo-2-(methylthio)pyrimidine-4-carboxylate (1.0 eq, 661 mg, 2.52 mmol) was dissolved in CH₂Cl₂ (10 ml). meta-chloro perbenzoic acid (m-cpba, 77% pure grade, 2.5 eq, 1.42 g, 6.34 mmol) was added and the mixture was stirred at room temperature for 1 hour. To the resulting suspension was added anhydrous THF (10 ml), methylamine hydrochloride (10 eq, 1.7 g, 25.18 mmol) and DIEA (10 eq, 4.3 ml, 24.69 mmol) and the mixture stirred at room temperature overnight. The solvents were removed in vacuo prior to adding CH₂Cl₂ and a saturated aqueous sodium bicarbonate solution. The two phases were decanted and two further CH₂Cl₂ extractions were carried out. The combined extracts were dried over Na₂SO₄ and the solvents evaporated. Purification by flash chromatography on silica gel (20-30% ethylacetate in hexanes) provided methyl 5-bromo-2-(methylamino)pyrimidine-4-carboxylate as an off-white solid (461 mg, 75% yield). LCMS (ES): >95% pure, m/z 246 [M]⁺, 248 [M+2]⁺.

### Process 9

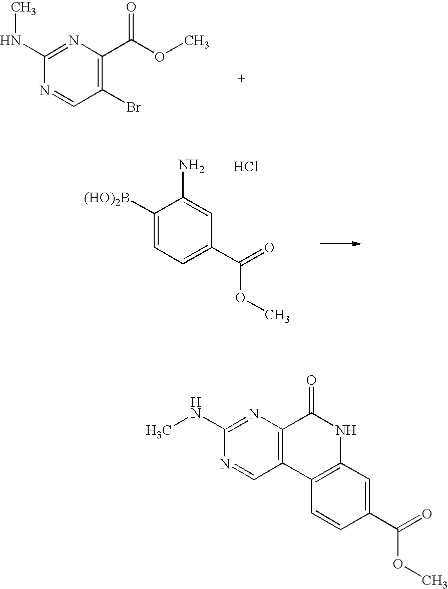

Sodium acetate (3.0 eq, 240 mg, 2.93 mmol) and 1,1′-bis(diphenylphosphino)ferrocene palladium (II) chloride (complexed with dichloromethane) (0.05 eq, 36 mg, 0.049 mmol) were added to a mixture of methyl 5-bromo-2-(methylamino)pyrimidine-4-carboxylate (1.0 eq, 240 mg, 0.975 mmol), and 2-amino-4-(methoxycarbonyl)phenylboronic acid hydrochloride (1.0 eq, 226 mg, 0.98 mmol) in anhydrous DMF (2 ml). The mixture was stirred under microwave heating at 120° C. for 10 min. Addition of water induced precipitation of the expected compound that was filtered and dried. methyl 3-(methylamino)-5-oxo-5,6-dihydropyrimido[4,5-c]quinoline-8-carboxylate (57 mg, 21% yield). LCMS (ES): >80% pure, m/z 285 [M+1]⁺.

### Process 10

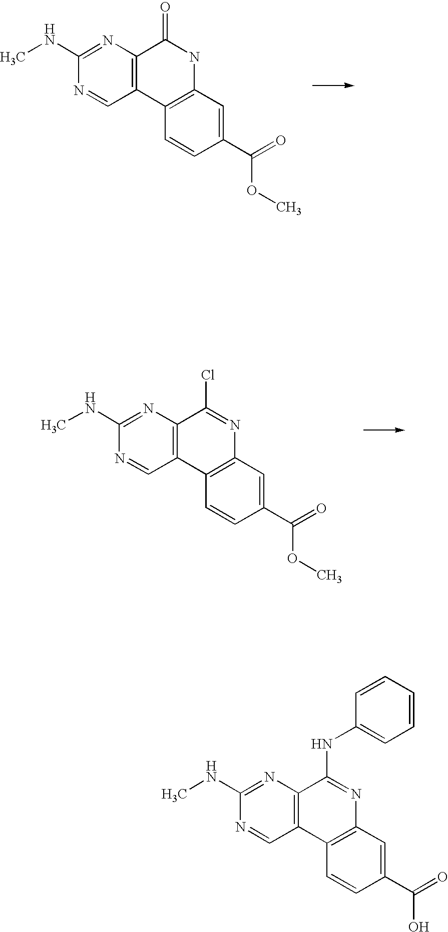

3-(methylamino)-5-(phenylamino)pyrimido[4,5-c]quinoline-8-carboxylic acid was prepared using methods described in process 3 and 4 starting from methyl 3-(methylamino)-5-oxo-5,6-dihydropyrimido[4,5-c]quinoline-8-carboxylate. The final product was purified by flash chromatography and isolated as a yellow solid (0.35 mg). LCMS (ES): >95% pure, m/z 346 [M+1]⁺.

### Process 11

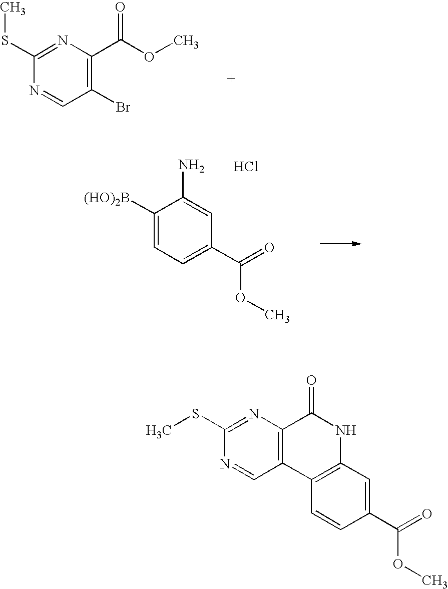

In a microwave vessel, methyl 5-bromo-2-(methylthio)pyrimidine-4-carboxylate (1.0 eq, 274 mg, 1.18 mmol), 2-amino-4-(methoxycarbonyl)phenylboronic acid hydrochloride (1.2 eq, 329 mg, 1.42 mmol), and sodium acetate (3.0 eq, 291 mg, 3.55 mmol) were mixed in anhydrous DMF (2 ml). The mixture was degassed by bubbling nitrogen gas in the solution for 10 min and the reaction heated under microwaves at 120° C. for 30 min. After cooling down the expected material crashed out of NMP. The solid was filtered, suspended in water filtered and dried. The material was triturated in AcOEt and filtered give a yellow solid. The same procedure was repeated 9 times using the same amounts of materials to provide methyl 3-(methylthio)-5-oxo-5,6-dihydropyrimido[4,5-c]quinoline-8-carboxylate (283 mg, 10% yield). LCMS (ES): >95% pure, m/z 302 [M+1]⁺, ¹H NMR (DMSO-d₆, 400 MHz) δ 2.71 (s, 3H), 3.89 (s, 3H), 7.80 (dd, J=1.6, J=8.4, 1H), 7.97 (d, J=1.6, 1H), 8.59 (d, J=8.8, 1H), 9.98 (s, 1H), 12.34 (s, 1H) ppm.

### Process 12

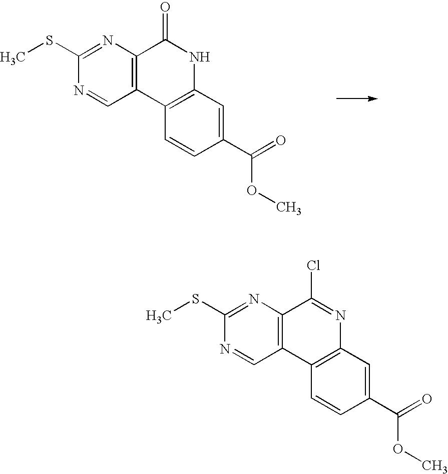

methyl 3-(methylthio)-5-oxo-5,6-dihydropyrimido[4,5-c]quinoline-8-carboxylate (1.0 eq, 279 mg, 0.926 mmol) was suspended in toluene (2 ml). POCl₃ (2 ml) and DIEA (0.5 ml) were added and the mixture stirred at 120° C. for 5 hours. The volatiles were removed in vacuo and CH₂Cl₂ was added. The organic phase was washed with saturated aqueous sodium bicarbonate, washed with water and dried over Na₂SO₄. The solution was filtered through a pad of celite and the solvents removed in vacuo. The material was triturated in hexanes and AcOEt, filtered and dried to provide methyl 5-chloro-3-(methylthio)pyrimido[4,5-c]quinoline-8-carboxylate as a beige solid (184 mg, 63% yield). LCMS (ES): >95% pure, m/z 320 [M+1]⁺, 322 [M+3]⁺.

### Process 13

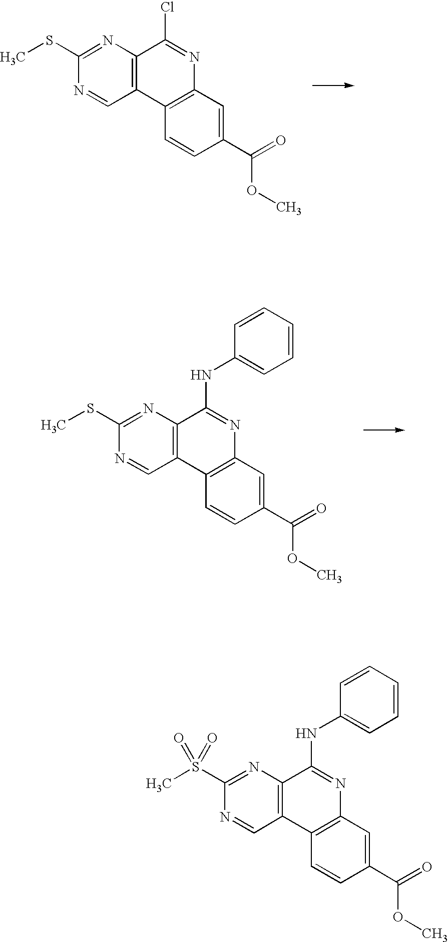

methyl 5-chloro-3-(methylthio)pyrimido[4,5-c]quinoline-8-carboxylate (1.0 eq, 182 mg, 0.57 mmol) was mixed with aniline (0.5 ml) in NMP (1 ml). The mixture was heated under microwave for 10 minutes at 120° C. Water was added and the resulting solid was filtered and dried. The compound was triturated in EtOAc and hexanes and filtered to afford methyl 3-(methylthio)-5-(phenylamino)pyrimido[4,5-c]quinoline-8-carboxylate as a yellow solid. LCMS (ES): >95% pure, m/z 377 [M+1]⁺. This material was suspended in CH₂Cl₂ (4 ml) and meta-chloroperbenzoic acid (77% pure, 2.5 eq, 165 mg, 0.737 mmol) was added in small portions. After one hour, an additional amount (100 mg) of mcpba was added and the mixture stirred for 1.5 hours. After addition of more CH₂Cl₂, the organic phase was washed with water (4×), dried over Na₂SO₄ and the solution was filtered through a pad of silica gel, eluting with a MeOH/CH₂Cl₂ mixture. After evaporation of the solvents, methyl 3-(methylsulfonyl)-5-(phenylamino)pyrimido[4,5-c]quinoline-8-carboxylate was isolated as a yellow solid (166 mg, 72% yield). LCMS (ES): >95% pure, m/z 409 [M+1]⁺, ¹H NMR (DMSO-d₆, 400 MHz) δ 3.77 (s, 3H), 3.93 (s, 3H), 7.15 (t, J=7.2, 1H), 7.45 (t, J=7.6, 2H), 7.99 (dd, J=2.0, J=8.4, 1H), 8.16 (d, J=7.6, 2H), 8.28 (d, J=2.0, 1H), 8.89 (d, J=8.8, 1H), 9.76 (s, 1H), 10.61 (s, 1H) ppm.

### Process 14

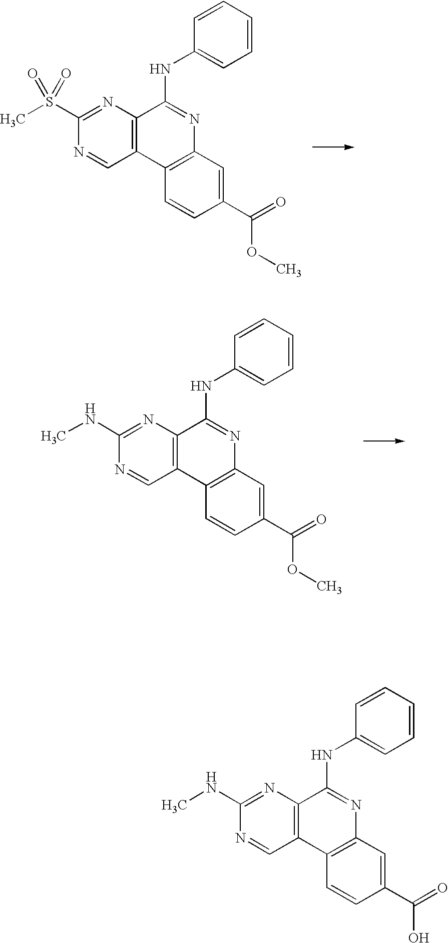

In a closed vial, methyl 3-(methylsulfonyl)-5-(phenylamino)pyrimido[4,5-c]quinoline-8-carboxylate (1.0 eq, 62 mg, 0.152 mmol) was mixed with Methylamine hydrochloride (100 mg), DIEA (260 ul) in DMF (1 ml). The mixture was stirred at 60° C. for 40 min. Addition of water induced precipitation of methyl 3-(methylamino)-5-(phenylamino)pyrimido[4,5-c]quinoline-8-carboxylate which was isolated by filtration. This material was suspended in a 1:1:1 mixture of THF, MeOH and water (4 ml), and vigorously stirred at 60° C. in the presence of LiOH (200 mg) for 1.5 hours. Water aqueous HCl were added and to reach pH=1. The solid was filtered, dried and triturated in AcOEt/hexanes to provide 3-(methylamino)-5-(phenylamino)pyrimido[4,5-c]quinoline-8-carboxylic acid as a yellow solid (40 mg, 74% yield). LCMS (ES): >95% pure, m/z 346 [M+1]⁺.

Other amines such as cyclopropylamine can be used in place of methylamine, and substituted anilines such as 3-(trifluoromethyl)aniline can be substituted for aniline to provide compounds such as the compound of formula (2) described herein. Synthesis of compound (2) produced a product which was characterized by the expected LC-MS.

The following examples are offered to illustrate but not to limit the invention.

### Evaluation of Pharmacokinetic Properties

The pharmacokinetics properties of drugs were investigated in three species, following an intravenous (IV) bolus or oral (PO) dose of Compound K at the dosages indicated in the chart. Blood samples were collected at predetermined times and the plasma separated. Plasma was separated from the blood samples collected at 5, 15 and 30 minutes and 1, 2, 4, 8 and 24 hours post-dose.

Drug levels were quantified by the LC/MS/MS method described below. Noncompartmental pharmacokinetic analysis was applied for intravenous administration. A linear trapezoidal rule was used to compute AUC(0-24). The terminal t_{1/2} and C₀ were calculated using the last three and the first three data points, respectively

Bioanalysis was performed using a Quattro Micro LC/MS/MS instrument in the MRM detection mode, with an internal standard (1S). Briefly, 15 μL plasma samples were prepared for analysis using protein precipitation with 120 μL of acetonitrile. The supernatants were transferred into a 96 well plate and subjected to LC-MS/MS analysis using a Phenomenex Polar-RP HPLC column. The mobile phases were 10 mM NH₄HCO₃ in water (Solution-A) and 10 mM NH₄HCO₃ in methanol (Solution-B). The column was initially equilibrated with 25% Solution-B and followed with 100% Solution B over 5 minutes. The method had a dynamic range from 1 to 10,000 ng/mL. Quantitation of the analytes was performed in the batch mode with two bracketing calibration curves according to the bioanalytical sample list.

| Species | Dose | | AUC_{0-inf} | Cₘₐₓ | CLₛ | Vdₛₛ | Terminal | |
|---|---|---|---|---|---|---|---|---|
| (n) | (mg/kg) | Route | (ng * hr/mL) | (ng/mL) | (L/hr/kg) | (L/kg) | T_{1/2} (hr) | % F_{0-inf} |
| Mouse | 5 | IV^{a} | 2004 | 6705 | 2.5 | 18.1 | 5.0 | |
| (4) | | | | | | | | |
| Mouse | 25 | PO^{b} | 2009 | 311 | | | 7.1 | 20.1 |
| (4) | | | | | | | | |
| Rat | 5.2 | IV^{a} | 44334 | 38683 | 0.1 | 2.0 | 11.6 | |
| (3) | | | | | | | | |
| Rat | 10.5 | PO^{b} | 42343 | 7369 | | | 12.3 | 47.8 |
| (3) | | | | | | | | |
| Dog | 1.9 | IV^{a} | 2541 | 5782 | 0.7 | 8.9 | 8.3 | |
| (3) | | | | | | | | |
| Dog | 8.2 | PO^{c} | 5646 | 2178 | | | 5.2 | 51.5 |
| (3) | | | | | | | | |
| Dog | 1.6 | IV^{a,d} | 1642 | 4438 | 0.9 | 11.3 | 8.3 | |
| (3) | | | | | | | | |
| Dog | 7.7 | PO^{c,d} | 3438 | 2.731 | | | 9.9 | 41.8 |
| (3) | | | | | | | | |

^{a}All IV tests used 25 mM sodium phosphate buffer as the vehicle
^{b}oral dosing for rodents used 25 mM phosphate buffer as the vehicle
^{c}oral dosing for dogs used filled capsules
^{d}These tests used a different lot of Compound K

### Example 1

### Pharmacokinetic Parameters for Compound 1

Using similar methods to those in Example 1, the pharmacokinetic behavior of Compound 1 was assessed in mice. A summary of the results is provided in the following table (Table 2).

TABLE 2

| PK Parameter | IV | PO | Unit |
|---|---|---|---|
| Dose | 5.4 | 10.8 | mg/kg |
| AUC_{(0-8 h)} | 11197.0 | 1052.9 | |
| AUC_{(0-24 h)} | 15305.3 | 2213.4 | ng · h · ml⁻¹ |
| AUC_{(0-Inf)} | 17029.4 | 3756.2 | ng · h · ml⁻¹ |
| Cmax-obs | 21846.1 | 800.0 | ng/mL |
| Cp0-exp | 29442.03 | N/A | ng/mL |
| Tmax | N/A | 0.25 | hr |
| Kel | 0.0678 | 0.0323 | hr⁻¹ |
| t_{1/2} | 10.22 | 21.47 | hr |
| Vd | 4.674 | N/A | L/kg |
| CLₛ | 0.317 | N/A | L/kg/hr |
| F_{(0-24 h)} | N/A | 7.2 | % |
| F_{(0-Inf)} | N/A | 11.0 | % |

### Example 2

### Inhibition of Pain in a Murine Model for Persistent Pain

Compounds K and (1) were tested in pain relief models using formalin-induced persistent pain. See Hunskaar, et al., “The formalin test in mice: Dissociation between inflammatory and non-inflammatory pain,” Pain, vol. 30, 103-114 (1987); Saddi, et al., “The formalin test in the mouse: a parametric analysis of scoring properties,” Pain, vol. 89, 53-63 (2000). These tests were conducted with ICR mice, in accordance with guidelines established by the International Association for the Study of Pain. Compounds of the invention were used as solutions of about 3-20 mg/mL in aqueous solution at about pH 8. In some instances, the compounds partially precipitated from the solution and were thus administered as suspensions. Persistent pain was induced by injecting 10 microliters of 2% formalin in saline solution, which was injected into the dorsal surface of the left hind paw. The test animals were monitored for at least 40 minutes, during which time the number of flinches occurring during each 5-minute period were observed and recorded. A cohort of 12 animals were used for each dose level of each compound tested. The observations of pain responses were done ‘blind’, meaning the observer did not know which animals had received which treatments. Statistical analysis of the results was done with Prism™ 5.0, using a one-way analysis of variance (ANOVA) for each compound versus vehicle; the level of significance was set at P<0.05.

The compounds were administered orally in a single dose, of 30, 100, and 200 mg/kg, one hour prior to injection of formalin Pain response was measured by flinching frequency. During the first 9 minute phase post-injection, neither compound affected rates of flinching. However, both compounds significantly reduced formalin-induced flinching during the 10-40 minute phase post-injection. Compound K was effective at the 30 and 200 mg/kg doses, and Compound (1) was effective at the 100 and 200 mg/kg dosages. A single dose of morphine (3 mg/kg, subcutaneously injected) served as a positive control, and reduced formalin-induced flinching in both phases of the test.

### Example 3

### Testing of Compounds in Anti-Hyperalgesia Model

Compound K and (1) were tested for efficacy in an inflammatory pain model, using CFA (Complete Freund's Adjuvant, 0.05% w/v mycobacterium Butyricum) to induce thermal hyperalgesia, as described by Hargreaves, et al. (Hargreaves, et al., “A new and sensitive method for measuring thermal nociception in cutaneous hyperalgesia,” Pain, vol. 32, 77-88 (1988).) ICR mice were used as discussed above, and each dosage of each compound was tested in at least 12 test animals. The observations of pain responses were done blindly. Statistical analysis of the results was done with Prism™ 5.0, using a one-way analysis of variance (ANOVA) for each compound versus vehicle; the level of significance was set at P<0.05. Both compounds precipitated from solution, so they were dosed as suspensions after vortexing and warming to promote solubility of the compound. Neither compound exhibited a statistically significant effect on CFA-induced thermal hyperalgesia at these doses. A single intraperitoneal dose of naproxen (50 mg/kg) as a positive control significantly reversed the CFA-induced thermal hyperalgesia at 1, 2, and 4 hours after naproxen administration.

The compounds were administered twice daily at a dose of 25, 75 or 150 mg/kg (BID) for four days. On day 4, test subjects received a single injection of CFA (20 microliters) subcutaneously in the plantar surface of the left rear paw. CFA was injected subcutaneously into the plantar surface of the left hind paw of each test subject, under isoflurane anesthesia. Inflammation at the site of injection was allowed to develop for about 24 hours before testing. On day 5, thermal responses were measured prior to treatment with the compound and again 1, 2, and 4 hours after treatment with CFA. Withdrawal latencies were measured in response to thermal stimulus by the Hargreaves method. Naproxen was used as a positive control.

### Example 5

### Phase I Clinical Study with Compound K

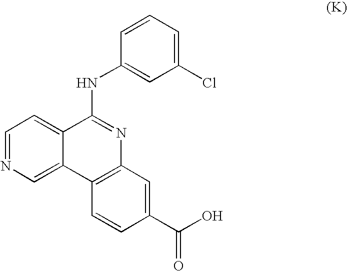

Compound K demonstrated single-agent potency in suppressing xenograft tumor growth with a wide therapeutic window pre-clinically. A Phase I study was undertaken to determine the maximum tolerated dose (MTD) and dose limiting toxicities (DLTs), to characterize the pharmacokinetics (PKs), and to study the pharmacodynamic effects of Compound K.

Procedure:

Eligible patients with advanced solid tumors, Castleman's disease or multiple myeloma with progressive disease, or for whom there are no available standard therapies, receive Compound K in successive dose cohorts at: 90, 160, 300, 460, 700 and 1000 mg per dose. Oral doses are administered twice daily for twenty-one consecutive days of a four week cycle. Therapy is continued in consenting patients until signs of intolerance to Compound K are observed, or there is evidence of advancing disease. Response by RECIST is determined after every 2 cycles. Serial blood and plasma samples are collected on the first and final dosing days of Cycle 1 (i.e., Day 1 and Day 21) for pharmacokinetic analysis and for pharmacodynamic biomarker evaluations (specifically, total and phosphorylated forms of p21 and Akt).

### Route and Schedule of Administration:

Patients in Cohorts 1-3 were dosed twice daily (BID) with oral capsules. Cohort 1 received 90 mg of Compound K BID. Cohort 2 received 160 mg of Compound K BID. Cohort 3 received 300 mg of Compound K BID.

### Summary of Results:

Thirteen patients with solid tumors (3-4 patients per cohort, from four separate dose cohorts) received oral doses of Compound K. These doses were well tolerated, with no drug-related significant adverse events of grade 3 or higher reported.

### Pharmacokinetic Analysis

Compound K demonstrated general linearity in PK parameters between the dose cohorts, with a terminal half life of approximately 25 hours at steady state.

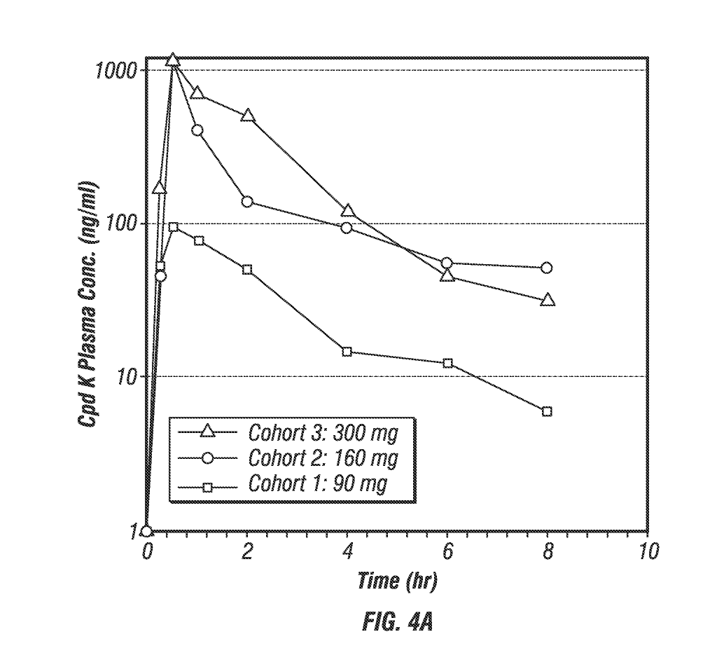

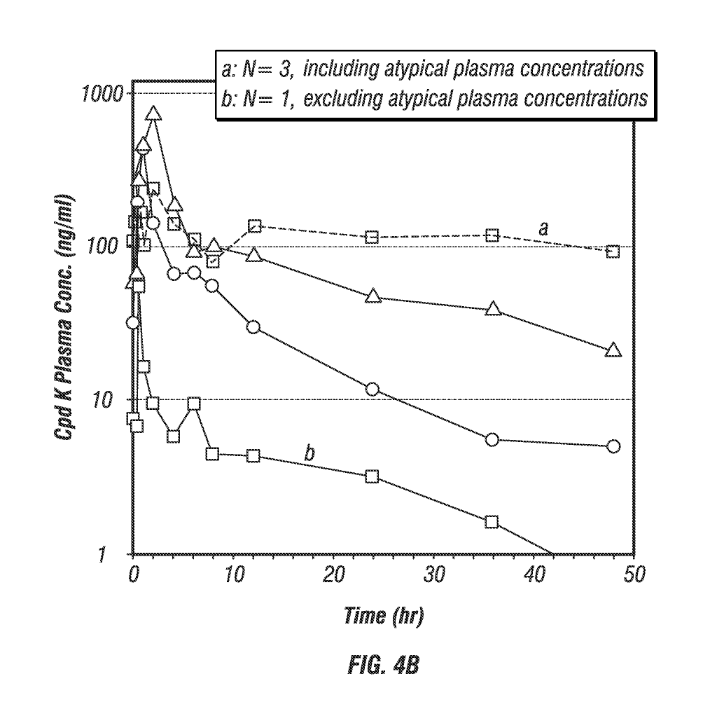

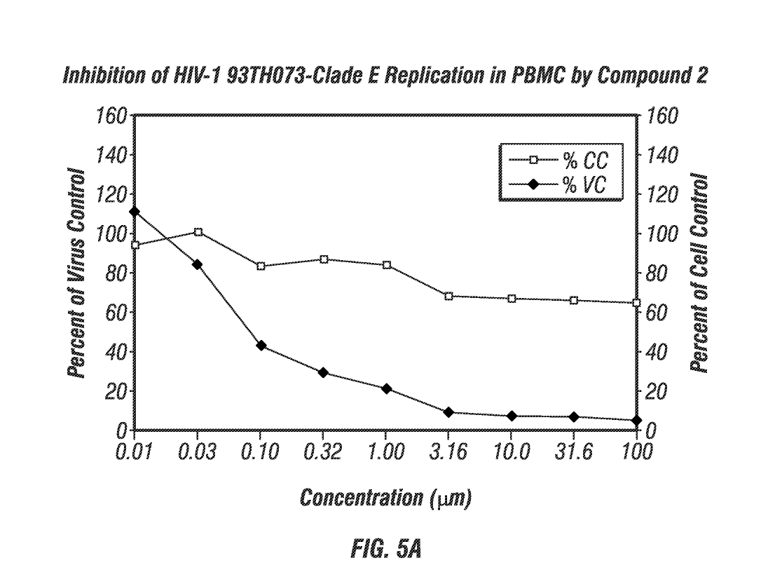

Plasma concentrations of Compound K were determined after the first dose on day 1 (FIG. 4A) and day 21 (FIG. 4B). Dose related plasma exposures were observed. No accumulation was observed.

### Conclusions:

Compound K has shown no drug related toxicity and has dose proportional pharmacokinetics. No dose-limiting toxicities (DLTs) have yet been observed, and the maximum tolerated dose (MTD) remains to be defined in this Phase I study. Further enrollment to the planned dose escalation cohorts is ongoing.

### Example 6

### CK2 Inhibitors in Antiviral Assays

Latent Infection Assay:

Histocytic leukemia cell line U937, latently infected with HIV-1, are treated with TNFα to induce virus expression in the presence of test compound. Antiviral activity is determined as a reduction in reverse transcriptase after 72 hr incubation.

PBMC Assay:

Acute infection of HIV-1 isolates (CXCR4-tropic HIV-1 Subtype B and CCR5-tropic HIV-1 Subtype) using fresh human PBMCs from multiple donors (PMA and IL-2 stimulated). Antiviral activity determined as a reduction in reverse transcriptase after 7-day incubation.

Data Analysis:

IC₅₀ (50% inhibition of virus replication)

TC₅₀ (50% host cell cytotoxicity)

TI=TC/IC; also referred to as Therapeutic Index values (TI) or Antiviral Index (AI)

### CK2 Inhibitors in U1 Latent Infection Assay

Compound K, Compound 1 and Compound 2 were tested in the U1 latent infection assay. The histocytic leukemia cell line U937, latently infected with HIV-1, was treated with TNFα to induce virus expression in the presence of test compound. Antiviral activity was determined as a reduction in reverse transcriptase after 72 hr incubation. Temacrazine, an inhibitor of HIV transcription initiation, was used as the positive control for the assay.

Compound K and Compound 1 showed no significant inhibition of virus production.

Compound 2 demonstrated an apparent biphasic effect that may be related to the effect of the compound on the cells. Compound 2 inhibited virus production at intermediate concentrations. Compound 2 appeared to enhance virus production from the U1 cells at high concentrations (e.g., potential stimulatory effect on virus transcription).

Data is shown in Table 3

TABLE 3
U1 Latent/Induced Antiviral Assay

| Compound | | | | |
|---|---|---|---|---|
| (Units) | IC₅₀ | IC₉₀ | TC₅₀ | TI (TC₅₀/IC₅₀) |
| Compound K Na | 0.70 | 18.0 | 10.6 | 15.2 |
| (μM) | | | | |
| Compound 1 Na | 0.20 | 9.08 | 17.1 | 87.3 |
| (μM) | | | | |
| Compound 2 Na | 3.24 | >100 | >100 | >30.9 |
| (μM) | 3.26 | >100 | >100 | >30.7 |
| Temacrazine | 3.56 | 17.9 | >100 | >28.1 |
| (nM) | 3.64 | 16.9 | >100 | >27.4 |

### CK2 Inhibitors in PBMC Assay:

Compound K, Compound 1 and Compound 2 were tested in the PBMC assay. Antiviral activity was determined as a reduction in reverse transcriptase after 7-day incubation. AZT, a nucleoside analog reverse transcriptase inhibitor, was used as the positive control for the assay.

Compound K and Compound 1 showed no significant inhibition of virus production.

Compound 2 inhibited virus production at intermediate concentrations. Data is shown in Table 4. 92HT599 is a CXCR4-tropic HIV-1 Subtype B virus. 91US005 is CCR5-tropic HIV-1 Subtype B virus.

TABLE 4
HIV-1 in PBMC's

| | | | | | TI |
|---|---|---|---|---|---|
| Compound (Units) | Virus | IC₅₀ | IC₉₀ | TC₅₀ | (TC₅₀/IC₅₀) |
| Compound K Na | 92HT599 | 3.49 | 8.25 | 7.61 | 2.18 |
| (μM) | 91US005 | 1.07 | 3.05 | | 7.13 |
| Compound 1 Na | 92HT599 | 1.15 | 2.77 | 3.53 | 3.07 |
| (μM) | 91US005 | 0.10 | 1.41 | | 35.8 |
| Compound 2 Na | 92HT599 | 4.52 | 20.6 | 12.0 | 2.65 |
| (μM) | 91US005 | 0.20 | 3.77 | | 59.6 |
| AZT | 92HT599 | 1.50 | 7.58 | >1,000 | >665 |
| (nM) | 91US005 | 1.64 | 6.86 | | >611 |

### CCR5—Tropic HIV-1 Clinical Isolates in Fresh Human PBMCs

Compound 2 was tested against eight CCR5-tropic HIV-1 clinical isolates in fresh human PBMCs. Compound 2 inhibited virus production in a dose-dependent manner in CCR5-Tropic HIV-1 infected PBMC's. Compound 2 had minimal cytotoxicity in PBMC's, providing a favorable therapeutic index. AZT was used as the positive control for the assay.

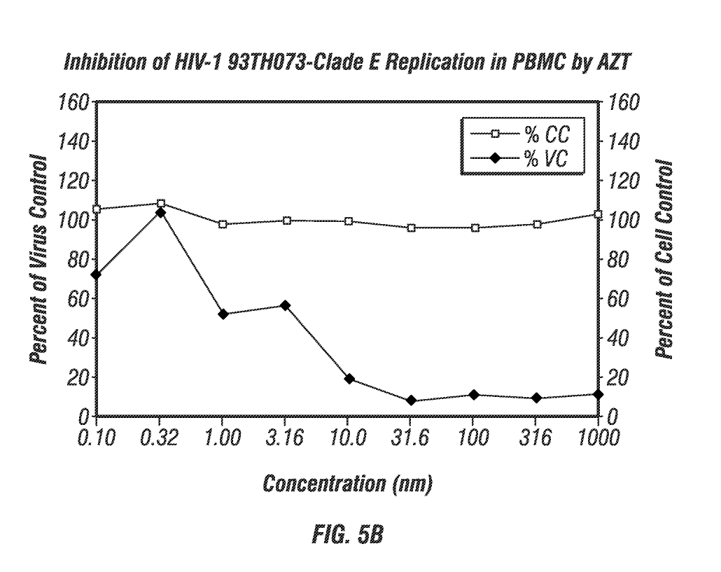

Data are provided in FIG. 5(A) for Compound 2 and in 5(B) for AZT.

### Example 7

### Representative Embodiments

A1. A method for treating or ameliorating a disorder other than a solid tumor that is associated with undesired activity of protein kinase CK2, which method comprises administering to a subject in need of such treatment or amelioration a therapeutically effective amount of a compound of formula (I):

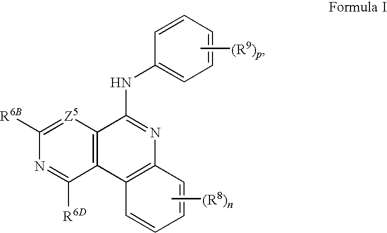

or a pharmaceutically acceptable salt or ester thereof,

wherein Z⁵ is N or CR^{6A};

each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R⁹ is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl, and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S;

n is 0 to 4; and

p is 0 to 4.

A2. A method for treating or ameliorating a disorder other than a solid tumor that is associated with undesired activity of protein kinase CK2, which method comprises administering to a subject in need of such treatment or amelioration a therapeutically effective amount of a compound having the formula:

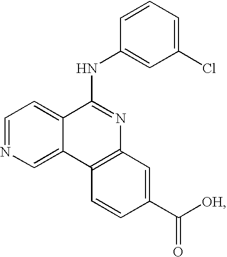

or a pharmaceutically acceptable salt or ester thereof.

A3. The method of embodiment A1 or A2, wherein said disorder is a neurodegenerative disorder, an inflammatory disorder, a disorder of the vascular system, a pathophysiological disorder of skeletal muscle or bone tissue, protozoan parasitosis, a viral disease, leukemia, lymphoma, and multiple myeloma.

A4. The method of embodiment A1 or A2, wherein said disorder is a neurodegenerative disorder.

A5. The method of embodiment A4, wherein said neurodegenerative disorder is Alzheimer's disease, Parkinson's disease, Guam-Parkinson dementia, chromosome 18 deletion syndrome, progressive supranuclear palsy, Kuf s disease, Pick's disease, memory impairment, or brain ischemia.

A6. The method of embodiment A1 or A2, wherein said disorder is an inflammatory disorder.

A7. The method of embodiment A6, wherein said inflammatory disorder is inflammatory pain, glomerulonephritis, multiple sclerosis, systemic lupus erythematosus, rheumatoid arthritis, or juvenile arthritis.

A8. The method of embodiment A1 or A2, wherein said disorder is a disorder of the vascular system.

A9. The method of embodiment A1 or A2, wherein said disorder of the vascular system is atherosclerosis, laminar shear stress or hypoxia.

A10. The method of embodiment A1 or A2, wherein said disorder is a pathophysiological disorder of skeletal muscle or bone tissue.

A11. The method of embodiment A10, wherein said pathophysiological disorder of skeletal muscle or bone tissue is cardiomyocyte hypertrophy, impaired insulin signaling or bone tissue mineralization.

A12. The method of embodiment A1 or A2, wherein said disorder is a protozoan parasitosis.

A13. The method of embodiment A1 or A2, wherein said disorder is a viral disease.

A14. The method of embodiment A13, wherein said viral disease is human immunodeficiency virus type 1 (HIV-1), human papilloma virus, or herpes simplex virus.

A15. The method of embodiment A1 or A2, wherein said disorder is leukemia, lymphoma or multiple myeloma.

A16. The method of any one of the embodiments claims, wherein said subject is human.

A17. A method for treating or ameliorating a disorder in a subject, which method comprises administering to said subject in need of such treatment or amelioration a therapeutically effective amount of a compound having the formula:

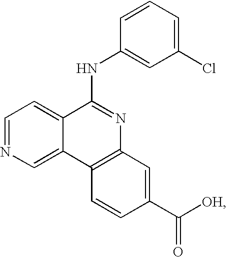

or a pharmaceutically acceptable salt or ester thereof;

wherein said disorder is selected from the group consisting of a neurodegenerative disorder, an inflammatory disorder, a disorder of the vascular system, a pathophysiological disorder of skeletal muscle or bone tissue, protozoan parasitosis, a viral disease, leukemia, lymphoma, and multiple myeloma.

A18. A method for treating or ameliorating a disorder in a subject, which method comprises administering to said subject in need of such treatment or amelioration a compound having the formula:

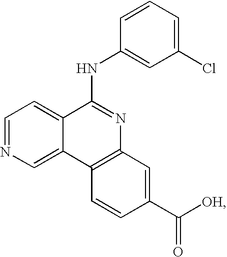

or a pharmaceutically acceptable salt or ester thereof,

in an amount effective to inhibit undesired activity of protein kinase CK2.

A19. The method of embodiment A18, wherein said disorder is selected from the group consisting of a neurodegenerative disorder, an inflammatory disorder, a disorder of the vascular system, a pathophysiological disorder of skeletal muscle or bone tissue, protozoan parasitosis, a viral disease, leukemia, lymphoma, and multiple myeloma.

A20. A method to treat pain, comprising administering to a subject in need of such treatment a compound of Formula I:

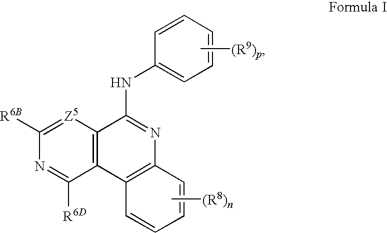

or a pharmaceutically acceptable salt or ester thereof,

wherein Z⁵ is N or CR^{6A};

each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S;

n is 0 to 4; and

p is 0 to 4.

A21. The method of embodiment A20, wherein the compound is a compound of Formula VIa:

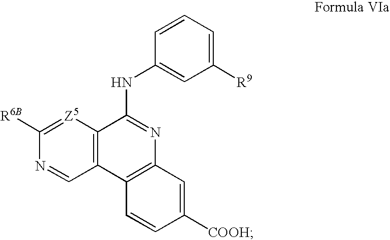

wherein R^{6B} can be H or —NHR′, where R′ is C1-C5 hydrocarbyl group, preferably C1-C3 alkyl or C3-C5 cycloalkyl; Z⁵ is CH or N; and R⁹ is halo, CF₃, or CCR″, where R″ is H or Me, or a pharmaceutically acceptable salt thereof.

A22. The method of embodiment A20 or A21, wherein the compound is selected from the group consisting of:

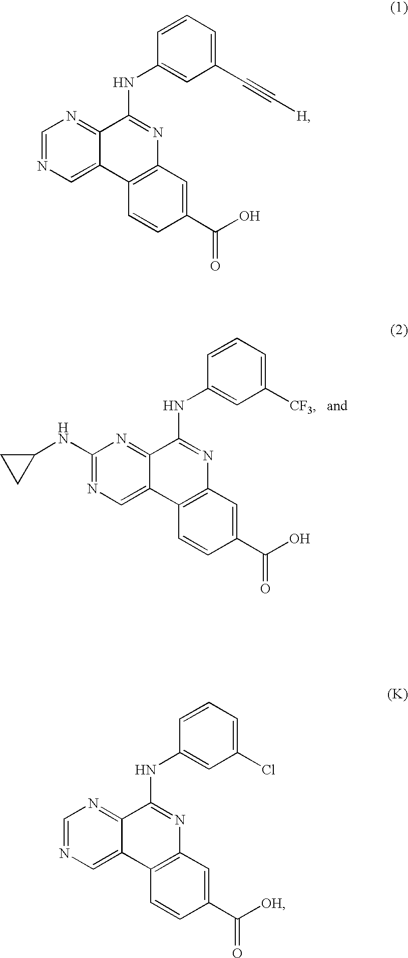

and the pharmaceutically acceptable salts and/or esters thereof.

A23. The method of embodiments A20, A21 or A22, wherein said pain is acute or chronic inflammatory pain.

A24. A method to treat a viral disease, comprising administering to a subject in need of such treatment a compound of Formula I:

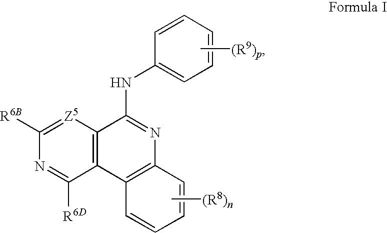

or a pharmaceutically acceptable salt or ester thereof,

wherein Z⁵ is N or CR^{6A};

each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S;

n is 0 to 4; and

p is 0 to 4.

A25. The method of embodiment A24, wherein the compound is a compound of Formula VIa:

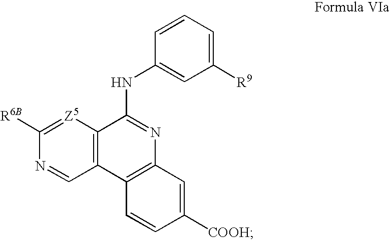

wherein R^{6B} can be H or —NHR′, where R′ is C1-C5 hydrocarbyl group, preferably C1-C3 alkyl or C3-C5 cycloalkyl; Z⁵ is CH or N; and R⁹ is halo, CF₃, or CCR″, where R″ is H or Me,

or a pharmaceutically acceptable salt thereof.

A26. The method of embodiment A24 or A25, wherein the compound is selected from the group consisting of:

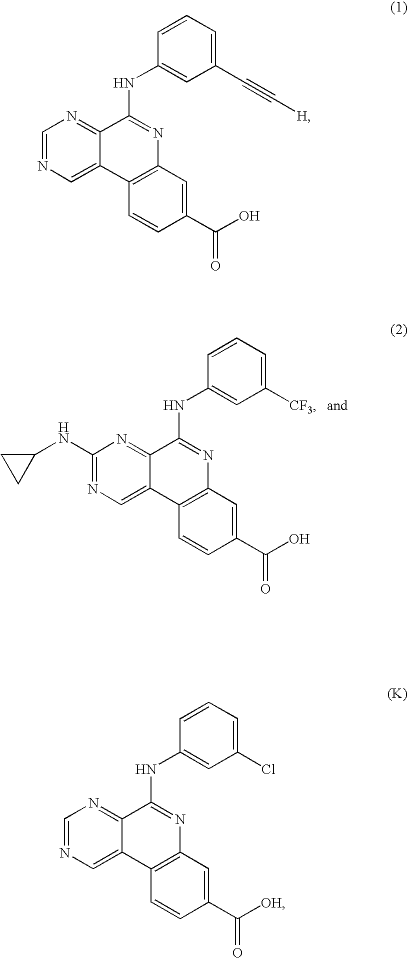

and the pharmaceutically acceptable salts and/or esters thereof.

A27. The method of embodiment A24, A25 or A26, wherein the viral disease is selected from the group consisting of human immunodeficiency virus type 1 (HIV-1), human papilloma virus (HPV), herpes simplex virus, Epstein-Barr virus, human cytomegalovirus, hepatitis C virus, hepatitis B virus, Borna disease virus, adenovirus, coxsackievirus, coronavirus, influenza, and varicella zoster virus.

A28. A method to treat an advanced solid tumor, comprising administering to a subject in need of such treatment a compound of Formula I:

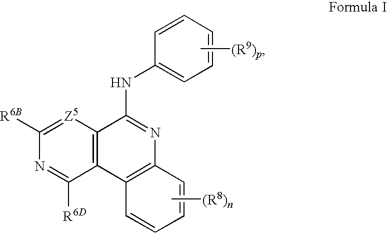

or a pharmaceutically acceptable salt or ester thereof,

wherein Z⁵ is N or CR^{6A};

each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S;

n is 0 to 4; and

p is 0 to 4.

A29. A method for treating, ameliorating or preventing a circadian rhythm disorder in a subject in need thereof, comprising administering to said subject a therapeutically effective amount of a compound of formula (I):

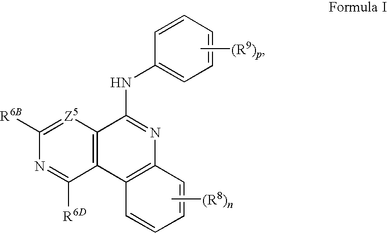

or a pharmaceutically acceptable salt or ester thereof,

wherein Z⁵ is N or CR^{6A};

each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S;

n is 0 to 4; and

p is 0 to 4.

A30. The method of embodiment A29, wherein the circadian rhythm disorder is selected from jet lag, shift work sleep disorder, delayed sleep phase syndrome (DSPS), advanced sleep phase syndrome, and non 24-hour sleep wake disorder.

A31. A method for modulating temperature compensation and/or circadian rhythm, which method comprises administering to a subject in need of such modulation a therapeutically effective amount of a compound of formula (I):

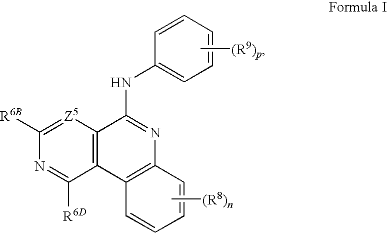

or a pharmaceutically acceptable salt or ester thereof,

wherein Z⁵ is N or CR^{6A};

each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′ SO₂R′, NR′ CONR′₂, NR′ COOR′, NR′ COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S;

n is 0 to 4; and

p is 0 to 4.

A32. The method of any one of embodiments A28, A29, A30, or A31, wherein the compound is a compound of Formula VIa:

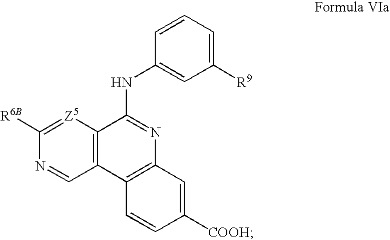

wherein R^{6B} can be H or —NHR′, where R′ is C1-C5 hydrocarbyl group, preferably C1-C3 alkyl

or C3-C5 cycloalkyl; Z⁵ is CH or N; and R⁹ is halo, CF₃, or CCR″, where R″ is H or Me,

or a pharmaceutically acceptable salt thereof.

A33. The method of any one of embodiments A28, A29, A30, A31 or A32, wherein the compound is selected from the group consisting of:

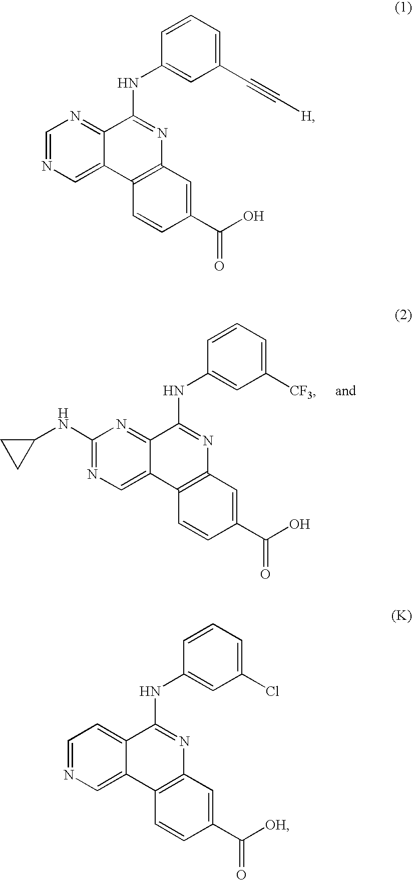

and the pharmaceutically acceptable salts and/or esters thereof.

The preceding examples and embodiments are provided to illustrate the invention and do not limit or define its scope. Suitable variations and alterations of these examples would be apparent to the person of ordinary skill in view of these examples and the description herein, and are included in the scope of the invention.

1. A method for treating or ameliorating a disorder other than a solid tumor that is associated with undesired activity of protein kinase CK2, which method comprises administering to a subject in need of such treatment or amelioration a therapeutically effective amount of a compound of formula (I): 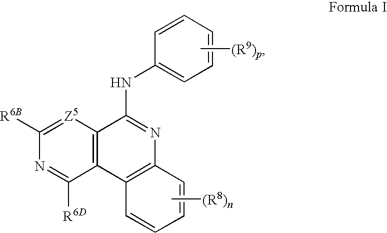
or a pharmaceutically acceptable salt or ester thereof,
wherein Z⁵ is N or CR^{6A};
each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOK, carboxy bioisostere, CONR₂, OOCR, COR, or NO₂,
each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or
each R⁹ is independently halo, OR, NR₂, NROR, NRNR₂, SR, SOR, SO₂R, SO₂NR₂, NRSO₂R, NRCONR₂, NRCOOR, NRCOR, CN, COOK, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S;
n is 0 to 4; and
p is 0 to 4.

2. The method of claim 1, wherein the compound is a compound of Formula VIa: 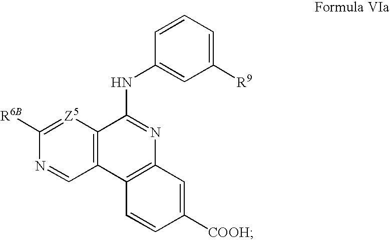
wherein R^{6B} can be H or —NHR′, where R′ is C1-C5 hydrocarbyl group, preferably C1-C3 alkyl
or C3-C5 cycloalkyl; Z⁵ is CH or N; and R⁹ is halo, CF₃, or CCR″, where R″ is H or Me,
or a pharmaceutically acceptable salt thereof.

3. The method of claim 1, wherein said disorder is a neurodegenerative disorder, an inflammatory disorder, a disorder of the vascular system, a pathophysiological disorder of skeletal muscle or bone tissue, protozoan parasitosis, a viral disease, leukemia, lymphoma, and multiple myeloma.

4. The method of claim 1, wherein said disorder is a neurodegenerative disorder.

5. The method of claim 4, wherein said neurodegenerative disorder is Alzheimer's disease, Parkinson's disease, Guam-Parkinson dementia, chromosome 18 deletion syndrome, progressive supranuclear palsy, Kuf's disease, Pick's disease, memory impairment, or brain ischemia.

6. The method of claim 1, wherein said disorder is an inflammatory disorder.

7. The method of claim 6, wherein said inflammatory disorder is inflammatory pain, glomerulonephritis, multiple sclerosis, systemic lupus erythematosus, rheumatoid arthritis, or juvenile arthritis.

8. The method of claim 1, wherein said disorder is a disorder of the vascular system.

9. The method of claim 8, wherein said disorder of the vascular system is atherosclerosis, laminar shear stress or hypoxia.

10. The method of claim 1, wherein said disorder is a pathophysiological disorder of skeletal muscle or bone tissue.

11. The method of claim 10, wherein said pathophysiological disorder of skeletal muscle or bone tissue is cardiomyocyte hypertrophy, impaired insulin signaling or bone tissue mineralization.

12. The method of claim 1, wherein said disorder is a protozoan parasitosis.

13. The method of claim 1, wherein said disorder is a viral disease.

14. The method of claim 13, wherein said viral disease is selected from the group consisting of human immunodeficiency virus type 1 (HIV-1), human papilloma virus (HPV), herpes simplex virus, Epstein-Barr virus, human cytomegalovirus, hepatitis C virus, hepatitis B virus, Borna disease virus, adenovirus, coxsackievirus, coronavirus, influenza, and varicella zoster virus.

15. The method of claim 1, wherein said disorder is leukemia, lymphoma or multiple myeloma.

16. A method to treat pain, comprising administering to a subject in need of such treatment a compound of Formula I: 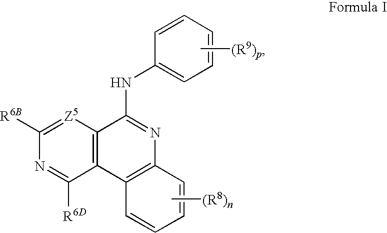
or a pharmaceutically acceptable salt or ester thereof,
wherein Z⁵ is N or CR^{6A};
each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or
each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S;
n is 0 to 4; and
p is 0 to 4.

17. The method of claim 16, wherein the compound is a compound of Formula VIa: 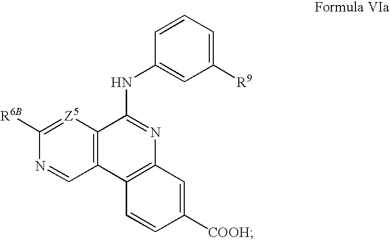
wherein R^{6B} can be H or —NHR′, where R′ is C1-C5 hydrocarbyl group, preferably C1-C3 alkyl or C3-C5 cycloalkyl; Z⁵ is CH or N; and R⁹ is halo, CF₃, or CCR″, where R″ is H or Me,
or a pharmaceutically acceptable salt thereof.

18. The method of claim 16, wherein said pain is acute or chronic inflammatory pain.

19. A method to treat an advanced solid tumor, comprising administering to a subject in need of such treatment a compound of Formula I: 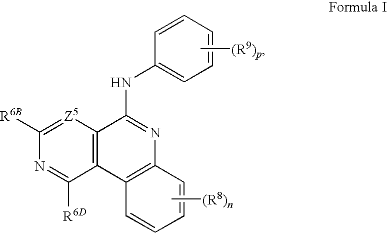
or a pharmaceutically acceptable salt or ester thereof,
wherein Z⁵ is N or CR^{6A};
each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or
each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S;
n is 0 to 4; and
p is 0 to 4.

20. A method for treating, ameliorating or preventing a circadian rhythm disorder in a subject in need thereof, comprising administering to said subject a therapeutically effective amount of a compound of formula (I): 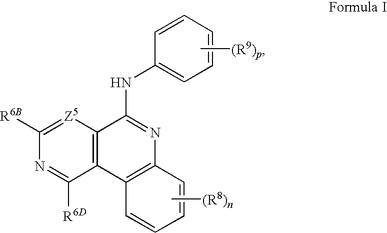
or a pharmaceutically acceptable salt or ester thereof,
wherein Z⁵ is N or CR^{6A};
each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or
each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S;
n is 0 to 4; and
p is 0 to 4.

21. The method of claim 20, wherein the circadian rhythm disorder is selected from jet lag, shift work sleep disorder, delayed sleep phase syndrome (DSPS), advanced sleep phase syndrome, and non 24-hour sleep wake disorder.

22. A method for modulating temperature compensation and/or circadian rhythm, which method comprises administering to a subject in need of such modulation a therapeutically effective amount of a compound of formula (1): 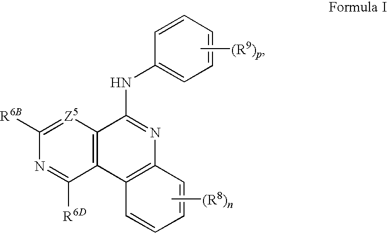
or a pharmaceutically acceptable salt or ester thereof,
wherein Z⁵ is N or CR^{6A};
each R^{6A}, R^{6B}, R^{6D} and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R^{6A}, R^{6B}, R^{6D} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or
each R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S;
n is 0 to 4; and
p is 0 to 4.
